(19) United States
(12) Patent Application Publication
(10) Pub. No.: US 2005/0245572 A1
(43) Pub. Date: Nov. 3, 2005

(54) Naphthyl ether compounds and their use

(76) Inventors: Peter Bernstein, Wilmington, DE (US); Joseph Cacciola, Wilmington, DE (US); Robert Dedinas, Wilmington (DE); Lihong Shen, Wilmington, DE (US); Paul Warwick, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/527,280

(22) PCT Filed: Sep. 8, 2003
(86) PCT No.: PCT/SE03/01399

§ 371 Date: Mar. 8, 2005

(30) Foreign Application Priority Data
Sep. 9, 2002 (SE) 0202674-8
Apr. 8, 2003 (SE) 0301052-7

(51) Int. Cl. C07D 211/18; A61K 31/445

(52) U.S. Cl. 514/317; 546/232

(57) ABSTRACT

Compounds having the following structure wherein R¹, R², R³, R⁴, R⁵, R⁶, R⁷ m and n are as defined in the specification, in vivo-hydrolysable precursors thereof, pharmaceutically-acceptable salts thereof, the use in therapy and pharmaceutical compositions and methods of treatment using the same.

### FIELD OF THE INVENTION

This invention relates to the treatment of diseases in which serotonin, Substance P or Neurokinin A are implicated, for example, in the treatment of disorders or conditions such as hypertension, depression, generalized anxiety disorder, phobias, posttraumatic stress syndrome, avoidant personality disorder, premature ejaculation, eating disorders, obesity, chemical dependencies, cluster headache, migraine, pain, Alzheimer's disease, obsessive-compulsive disorder, panic disorder, memory disorders, Parkinson's disease, endocrine disorders vasospasm, cerebellar ataxia, gastrointestinal tract disorders, negative symptoms of schizophrenia, premenstrual syndrome, fibromyalgia syndrome, stress incontinence, Tourette's syndrome, trichotillomania, kleptomania, male impotence, attention deficit hyperactivity disorder, chronic paroxysmal hemicrania and headache.

### BACKGROUND

The mammalian neurokinins are peptide neurotransmitters found in the peripheral and central nervous systems. The three principal neurokinins are Substance P(SP), Neurokinin A (NKA) and Neurokinin B (NKB). N-terminally extended forms of at least NKA are known.

Three receptor types are known for the principal neurokinins. Based upon their relative selectivities for the neurokinins SP, NKA and NKB, the receptors are classified as neurokinin 1 (NK₁), neurokinin 2 (NK₂) and neurokinin 3 (NK₃) receptors, respectively. In the periphery, SP and NKA are localized in C-afferent sensory neurons, which neurons are characterized by non-myelinated nerve endings known as C-fibers, and are released by selective depolarization of these neurons, or selective stimulation of the C-fibers. C-Fibers are located in the airway epithelium, and the tachykinins are known to cause profound effects which clearly parallel many of the symptoms observed in asthmatics. The effects of release or introduction of tachykinins in mammalian airways include bronchoconstriction, increased microvascular permeability, vasodilation, increased mucus secretion and activation of mast cells. Neurokinin antagonists that interact with NK₁, NK₂ and NK₃ receptors, having different chemical structures have been described. Particularly international publications WO 98/07722, WO 96/39383 and WO 98/25617, and regional publications EP 428434, EP 474561, EP 515240 and EP 559538 disclose the preparation of a variety of chemical structures.

NK₁ activity is also implicated in depression and anxiety, mice with genetically altered NK, receptors have decreased anxiety related behavior (Santarelli, L., et. al., Proc. Nat. Acad. Sci., 98, 1912 (2001)) and NK₁ antagonists have been reported to be effective in an animal model of depression (Papp, M., et. al., Behav. Brain Res., 115, 19 (2000)).

Serotonin Selective Reuptake Inhibitors (SSRIs) are widely used for the treatment of major depressive disorder (MDD) and are considered well-tolerated and easily administered. SSRIs, however, have a delayed onset of action, are associated with undesirable side effects such as sexual dysfunction, and are ineffective in perhaps 30% of patients (M. J. Gitlin, J. Clin. Psych., 55, 406413 (1994)).

Compounds with dual action as NK₁ antagonists and serotonin reuptake inhibitors may, therefore provide a new class of antidepressants. Indeed, compounds combining NK₁ antagonism and serotonin reuptake inhibition have been described (Ryckmans, T., et. al., Bioorg. Med. Chem. Lett., 12, 261 (2002))

### SUMMARY OF THE INVENTION

We have discovered substituted naphthyl ether derivatives having both neurokinin 1 (NK₁) antagonist activity and serotonin reuptake inhibitory (SRI) activity. Naphthyl ether derivatives of the invention are compounds in accord with formula I: 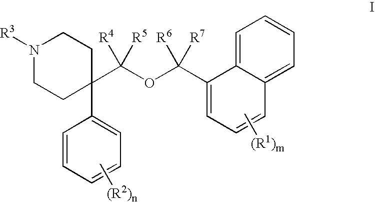
wherein:
R¹ at each occurrence is a moiety independently selected from CN, CF₃, OCF₃, OCHF₂, halogen, C₁₋₄alkyl, C₂₋₄alkenyl, C₂₋₄alkynyl, R^{a}, R^{b}, SR^{a}, NR^{e}R^{f}, CH₂NR^{e}R^{f}, OR^{c}, and CH₂OR^{c}, where m is selected from 0, 1, 2 or 3; wherein R^{a}, R^{b}, and R^{c} are independently at each occurrence selected from hydrogen, C₁₋₆alkyl, C(O)R^{d}, C(O)NHR^{d} and CO₂R^{d}, or R^{a} and R^{b} may together be (CH₂)ⱼG(CH₂)ₖ or G(CH₂)ⱼG where G is oxygen, j is 1, 2, 3 or 4, k is 0, 1 or 2; where R^{d} at each occurrence is independently selected from C₁₋₆alkyl, and R^{e} and R^{f} are independently at each occurrence selected from hydrogen, C₁₋₆alkyl, C(O)R^{d}, C(O)NHR^{d}, CO₂R^{d};
R² at each occurrence is independently selected from hydrogen, CN, CF₃, OCF₃, OCHF₂, halogen, C₁₋₄alkyl, C₂₋₄alkenyl, C₂₋₄alkynyl, R^{a}, R^{b}, SR^{a}, NR^{e}R^{f}, CH₂NReR, OR^{c}, and CH₂OR^{c}, where n is selected from 0, 1, 2 or 3; wherein R^{a}, R^{b}, and R^{c} are independently at each occurrence selected from hydrogen, C₁₋₆alkyl, C(O)R^{d}, C(O)NHR^{d} and CO₂R^{d}, or R^{a} and R^{b} may together be (CH₂)ⱼG(CH₂)ₖ or G(CH₂)ⱼG where G is oxygen, j is 1, 2, 3 or 4, k is 0, 1 or 2; where R^{d} at each occurrence is independently selected from C₁₋₆alkyl, and R^{e} and R^{f} are independently at each occurrence selected from hydrogen, C₁₋₆alkyl, C(O)R^{d}, C(O)NHR^{d}, CO₂R^{d};
R³ is selected from hydrogen, C₁₋₆alkyl, C(O)—(CH₂)_{q}—NR⁸R⁹, (CH₂)ₙ—NR⁸R⁹, (CH₂)_{q}—O-D, (CH₂)_{q}-D and (CH₂)_{q}—CH═CH-D, wherein R⁸ and R⁹ are independently selected from hydrogen and C₁₋₆alkyl, q is selected from 1, 2 or 3, r is selected from 1, 2, 3 or 4 and D is selected from phenyl or indolyl which phenyl or indolyl may bear one or more substituents selected from halogen, C₁₋₆alkyl, C₁₋₆alkoxy and —O—(CH₂)_{q}—O—;
R⁴, R⁵, R⁶ and R⁷ at each occurrence are independently selected from hydrogen or C₁₋₆alkyl, or
independently, R⁴ and R⁵ together with the carbon to which they are attached and R⁶ and R⁷ together with the carbon to which they are attached form a moiety in accord with formula II, 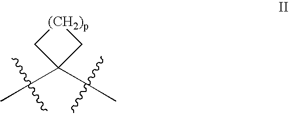
   wherein p is selected from 0, 1, 2, 3 or 4.

The invention also encompasses in vivo-hydrolysable precursors and pharmaceutically-acceptable salts of the naphthyl ether derivatives, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

### DETAILED DESCRIPTION OF THE INVENTION

This invention comprises novel naphthyl ether derivatives having dual NK₁ antagonist activity and SRI activity, pharmaceutical compositions containing such compounds and methods of using such compounds to treat central nervous system (CNS) and other disorders.

Compounds of the present invention are those in accord with formula I: 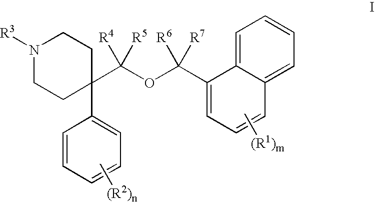
wherein:
R¹ at each occurrence is a moiety independently selected from CN, CF₃, OCF₃, OCHF₂, halogen, C₁₋₄alkyl, C₂₋₄alkenyl, C₂₋₄alkynyl, R^{a}, R^{b}, SR^{a}, NR^{e}R^{f}, CH₂NR^{e}R^{f}, OR^{c}, and CH₂OR^{c}, where m is selected from 0, 1, 2 or 3; wherein R^{a}, R^{b}, and R^{c} are independently at each occurrence selected from hydrogen, C₁₋₆alkyl, C(O)R^{d}, C(O)NHR^{d} and CO₂R^{d}, or R^{a} and R^{b} may together be (CH₂)ⱼG(CH₂)ₖ or G(CH₂)ⱼG where G is oxygen, j is 1, 2, 3 or 4, k is 0, 1 or 2; where R^{d} at each occurrence is independently selected from C₁₋₆alkyl, and R^{e} and R^{f} are independently at each occurrence selected from hydrogen, C₁₋₆alkyl, C(O)R^{d}, C(O)NHR^{d}, CO₂R^{d};
R² at each occurrence is independently selected from hydrogen, CN, CF₃ OCF₃, OCHF₂, halogen, C₁₋₄alkyl, C₂₋₄alkenyl, C₂₋₄alkynyl, R^{a}, R^{b}, SR^{a}, NR^{e}R^{f}, CH₂NR R^{f}, OR^{c}, and CH₂OR^{c}, where n is selected from 0, 1, 2 or 3; wherein R^{a}, R^{b}, and R^{c} are independently at each occurrence selected from hydrogen, C₁₋₆alkyl, C(O)R^{d}, C(O)NHR^{d} and CO₂R^{d}, or R^{a} and R^{b} may together be (CH₂)ⱼG(CH₂)ₖ or G(CH₂)ⱼG where G is oxygen, j is 1, 2, 3 or 4, k is 0, 1 or 2; where R^{d} at each occurrence is independently selected from C₁₋₆alkyl, and R^{e} and R^{f} are independently at each occurrence selected from hydrogen, C₁₋₆alkyl, C(O)R^{d}, C(O)NHR^{d}, CO₂R^{d};
R³ is selected from hydrogen, C₁₋₆alkyl, C(O)—(CH₂)_{q}—NR⁸R⁹, (CH₂)ᵣ—NR⁸R⁹, (CH₂)_{q}—O-D, (CH₂)_{q}-D and (CH₂)_{q}—CH═CH-D, wherein R⁸ and R⁹ are independently selected from hydrogen and C₁₋₆alkyl, q is selected from 1, 2 or 3, r is selected from 1, 2, 3 or 4 and D is selected from phenyl or indolyl which phenyl or indolyl may bear one or more substituents selected from halogen, C₁₋₆alkyl, C₁₋₆alkoxy and —O—(CH₂)_{q}—O—;
R⁴, R⁵, R⁶ and R⁷ at each occurrence are independently selected from hydrogen or C₁₋₆alkyl, or
independently, R⁴ and R⁵ together with the carbon to which they are attached and R⁶ and R⁷ together with the carbon to which they are attached form a moiety in accord with formula II, 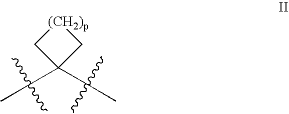
   wherein p is selected from 0, 1, 2, 3 or 4, in vivo-hydrolysable precursors thereof, and pharmaceutically-acceptable salts thereof.

Particular compound of the invention are those wherein:
R¹ at each occurrence is independently selected from fluoro, cyano, C₁₋₆alkyl and C₁₋₆alkoxy and m is 1, 2 or 3;
R² at each occurrence is independently selected from halogen where n is 1 or 2, and
R³ is selected from hydrogen and C₁₋₆alkyl; in vivo-hydrolysable precursors thereof, and pharmaceutically-acceptable salts thereof.

Other particular compound of the invention are those wherein:
R¹ at each occurrence is independently selected from fluoro, cyano, C₁₋₆alkyl and C₁₋₆alkoxy and m is 1, 2 or 3;
R² at each occurrence is independently selected from halogen where n is 1 or 2, and
R³ is selected from hydrogen, C₁₋₆alkyl, C(O)—(CH₂)_{q}—NR⁸R⁹, (CH₂)ₙ—NR⁸R⁹, (CH₂)_{q}-O-D, wherein R⁸ and R⁹ are independently selected from hydrogen, C₁₋₆alkyl and C₁₋₆alkoxy, q is 1, 2 or 3, r is 1, 2, 3 or 4 and D is selected from phenyl, indol-3-yl, indol-4-yl which phenyl may bear one or more substituents selected from fluoro, methyl, ethyl, methoxy, ethoxy or —O—(CH₂)₂—O— and which indolyl may bear one or more substituents selected from fluoro, methyl, ethyl, methoxy or ethoxy, in vivo-hydrolysable precursors thereof, and pharmaceutically-acceptable salts thereof.

More particular compound of the invention are those wherein:
R¹ at each occurrence is independently selected from fluoro, cyano, ethyl and methoxy and m is 1, 2 or 3;
R² at each occurrence is independently selected from halogen where n is 1 or 2, and
R³ is selected from hydrogen and methyl; in vivo-hydrolysable precursors thereof, and pharmaceutically-acceptable salts thereof.

Particular compounds of the invention are compounds wherein R⁴, R⁵ and R⁶ are each hydrogen and R⁷ is methyl and pharmaceutically-acceptable salts thereof.

Most particular compounds of the invention are those of the examples herein.

Pharmaceutically-acceptable salts of compounds in accord with formula I include those made with inorganic or organic acids which afford a physiologically-acceptable anion, such as with, for example, hydrochloric, hydrobromic, sulfuric, phosphoric, methanesulfonic, sulfamic, para-toluenesulfonic, acetic, citric, lactic, tartaric, malonic, fumaric, ethanesulfonic, benzenesulfonic, cyclohexylsulfamic, salicyclic and quinic acids.

Yet a further aspect of the present invention is a method of treating a disease condition wherein antagonism of NK₁ receptors in combination with SRI activity is beneficial which method comprises administering to a warm-blooded animal an effective amount of a compound in accord with formula I or an in vivo-hydrolysable precursor or a pharmaceutically-acceptable salt thereof. The present invention also provides the use of a compound in accord with formula I or an in vivo-hydrolysable precursor or a pharmaceutically-acceptable salt thereof in the preparation of a medicament for use in a disease condition wherein antagonism of the NK₁ receptors and SRI activity is beneficial.

The present invention also relates to a method for treating a disorder or condition selected from hypertension, depression in cancer patients, depression in Parkinson's patients, postmyocardial infarction depression, subsyndromal symptomatic depression, depression in infertile women, pediatric depression, major depression, single episode depression, recurrent depression, child abuse induced depression, post partum depression, generalized anxiety disorder, agoraphobia, social phobia, simple phobias, posttraumatic stress syndrome, avoidant personality disorder, premature ejaculation, anorexia nervosa, bulimia nervosa, obesity, addictions to alcohol, cocaine, heroin, phenobarbital, nicotine or benzodiazepines; cluster headache, migraine, pain, Alzheimer's disease, obsessive-compulsive disorder, panic disorder, dementia, amnestic disorders, age-related cognitive decline, dementia in Parkinson's disease, neuroleptic-induced parkinsonism, tardive dyskinesias, hyperprolactinaemia, vasospasm, cerebral vasculature vasospasm, cerebellar ataxia, gastrointestinal tract disorders, negative symptoms of schizophrenia, premenstrual syndrome, fibromyalgia syndrome, stress incontinence, Tourette's syndrome, trichotillomania, kleptomania, male impotence, attention deficit hyperactivity disorder, chronic paroxysmal hemicrania and headache associated with vascular disorders in a mammal, comprising administering an effective amount of a compound in accord with formula I or a pharmaceutically-acceptable salt thereof effective in treating such disorder or condition and a pharmaceutically-acceptable carrier.

The present invention also relates to a pharmaceutical composition for treating a disorder or condition selected from hypertension, depression (e.g., depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, attention deficit hyperactivity disorder (ADHD), chronic paroxysmal hemicrania and headache (associated with vascular disorders) in a mammal, preferably a human, comprising an effective amount of a compound in accord with formula I or a pharmaceutically-acceptable salt thereof effective in treating such disorder or condition and a phammaceutically-acceptable carrier.

In another aspect the invention relates to compounds according to formula I and their use in therapy and compositions containing them.

In a further aspect the invention relates to compounds according to formula I wherein one or more of the atoms is labeled as a radioisotope of the same element. In a particular form of this aspect of the invention the compound of formula I is labeled with tritium.

Compounds of the invention labeled with tritium are useful for the discovery of novel medicinal compounds which bind to and modulate the activity of the NK1 and SRI receptors. Such tritium-labelled compounds may be used in assays that measure the displacement of a such compounds to assess the binding of ligands that bind to NK1 or SRI receptors.

In a particular aspect the invention relates to the use of compounds according to formula I for the therapy of diseases mediated through the action of NK1 and SRI receptors. A more particular aspect of the invention relates to the use of compounds of formula I for the therapy of diseases mediated through the action of NK1 and SRI receptors.

Another aspect of the invention relates to a method of treatment or prophylaxis of human diseases or conditions in which activation of the NK1 and SRI receptors is beneficial which comprises administering a therapeutically effective amount of a compound of the invention.

A particular embodiment of this aspect of the invention relates to methods of treating conditions selected from hypertension, depression (e.g., depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, attention deficit hyperactivity disorder (ADHD), chronic paroxysmal hemicrania and headache (associated with vascular disorders) in a mammal, preferably a human, comprising administering an effective amount of a compound in accord with formula I or a pharmaceutically-acceptable salt thereof effective in treating such disorder or condition.

Another aspect of the invention relates to a pharmaceutical composition comprising a compound of the invention and a pharmaceutically-acceptable diluent or carrier.

A further aspect of the invention relates to a pharmaceutical composition for treating or preventing a condition or disorder mentioned herein arising from dysfunction of NK1 and SRI receptors in a mammal, preferably a human, comprising an amount of a compound of formula I, an enantiomer thereof or a pharmaceutically acceptable salt thereof, effective in treating or preventing such disorder or condition and a pharmaceutically acceptable carrier.

Another aspect of the invention relates to use of the pharmaceutical composition of the invention for the treatment of prophylaxis of human diseases or conditions in which activation of the NK1 and SRI receptors is beneficial.

Another aspect of the invention relates to use of the pharmaceutical composition of the invention for the treatment or prophylaxis of neurological disorders, psychotic disorders or intellectual impairment disorders.

Another aspect of the invention relates to a use of a compound of the invention in the manufacture of a medicament for the treatment or prophylaxis of human diseases or conditions in which activation of the NK1 and SRI receptors is beneficial.

Another aspect of the invention relates to a use of a compound of the invention in the manufacture of a medicament for the treatment or prophylaxis of neurological disorders, psychotic disorders or intellectual impairment disorders.

A particular embodiment of this aspect of the invention relates to the use of a compound of the invention in the manufacture of a medicament for treatment or prophylaxis of hypertension, depression (e.g., depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, attention deficit hyperactivity disorder (ADHD), chronic paroxysmal hemicrania and headache (associated with vascular disorders) in a mammal, preferably a human, comprising an effective amount of a compound in accord with formula I or a pharmaceutically-acceptable salt thereof effective in treating such disorder or condition and a pharmaceutically-acceptable carrier.

For the uses, methods and compositions mentioned herein the dosage administered will, of course, vary with the compound employed, the mode of administration and the treatment desired. However, in general, satisfactory results are obtained when the compounds of the invention are administered at a daily dosage of from about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carrier or diluent.

The compounds of formula I, an enantiomer thereof, and pharmaceutically acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically acceptable diluent or carrier.

In order to use a compound in accord with formula I or an in vivo-hydrolysable precursor or a pharmaceutically-acceptable salt thereof for the therapeutic treatment or prophylactic treatment of mammals including humans, it is normally formulated in accordance with standard pharmaceutical practice as a pharmaceutical composition. Therefore, another aspect the present invention is a pharmaceutical composition comprising a compound in accord with formula I, an in vivo-hydrolysable precursor or a pharmaceutically-acceptable salt thereof and a pharmaceutically-acceptable carrier.

Pharmaceutical compositions of this invention may be administered in standard manner for the disease condition that it is desired to treat, for example by oral, topical, parenteral, buccal, nasal, vaginal or rectal administration or by inhalation or insufflation. For these purposes the compounds of this invention may be formulated as tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions and in other forms as will be known to those of skill in the art.

In addition to the compounds of the present invention the pharmaceutical composition of this invention may also contain, or be co-administered (simultaneously or sequentially) with, one or more pharmacological agents of value in treating one or more disease conditions referred to herein.

The pharmaceutical compositions of this invention will normally be administered to humans so that a daily dose of 0.01 to 25 mg/kg body weight (and preferably of 0.1 to 5 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention. A tablet or capsule for oral administration may conveniently contain up to 250 mg (and typically 5 to 100 mg) of a compound in accord with formula I or a pharmaceutically-acceptable salt thereof. In another example, for administration by inhalation, a compound in accord with formula I or an in vivo-hydrolysable precursor or a pharmaceutically-acceptable salt thereof may be administered in a daily dosage range of 5 to 100 mg, in a single dose or divided into two to four daily doses. In a further example, for administration by intravenous or intramuscular injection or infusion, a sterile solution or suspension containing up to 10% w/w (and typically 5% w/w) of a compound in accord with formula I or an in vivo-hydrolysable precursor or a pharmaceutically-acceptable salt thereof may be used.

Compounds in accord with formula I and their in vivo-hydrolysable precursors or a pharmaceutically-acceptable salts may be made by processes as described and exemplified herein and by processes similar thereto and by processes known in the chemical art. If not commercially available, starting materials for these processes may be made by procedures which are selected from the chemical art using techniques which are similar or analogous to the synthesis of known compounds.

Pharmaceutically-acceptable salts may be prepared from the corresponding acid in a conventional manner. Non-pharmaceutically-acceptable salts may be useful as intermediates and as such are another aspect of the present invention.

It is well known in the art how to prepare optically-active forms (for example, by resolution of the racemic form or by synthesis from optically-active starting materials) and all optically active forms, enantiomers are compounds of this invention.

The following biological test methods, data and Examples serve to illustrate and further describe the invention.

The utility of a compound of the invention or an in vivo-hydrolysable precursor or a pharmaceutically-acceptable salt thereof (hereinafter, collectively referred to as a “Compound”) may be demonstrated by standard tests and clinical studies, including those disclosed in the publications described below.

Biological Assays:

Test A: SERT Binding Assay:

Frozen membrane preparations of a stably transfected HEK293 cell line expressing human 5-HTT receptors were purchased from Receptor Biology (PerkinElmer). Frozen alliquots were rapidly thawed, homogenized, and diluted in assay buffer (AB) containing 50 mM TRIS-HCL, 120 mM NaCl, 5 mM KCl and adjusted to pH 7.4 with NaOH. Final protein concentration was 40 μg/mL. Test compounds were evaluated in competition assays utlilizing [³H]-lmipramine Hydrochloride purchased from NEN (PerkinElmer) as the radioligand. The stock radioligand was diluted with AB for a final concentration of approximately 2 nM. Kd for [³H]-Imipramine Hydrochloride was determined to be 2.7 nM. The competition assays were performed on 96-well assay plates—two drugs per plate. Ten serial dilutions (normally 1 μM to 38 μM final concentration) from stock 10 mM solutions of compounds prepared in DMSO. All serial dilutions were made using 20% DMSO. DMSO content in assay is less than 1%. Incubation mixtures were prepared in quadruplicate in 96-well plates (Costar). Final assay volumes per well were 10 μl compound/nonspecific/control (1% DMSO), 20 μl membranes, 20 μl [³H]-Imipramine Hydrochloride, and 150 μl AB. Specific binding was defined by using 10 μM Imipramine. The binding reaction was initiated by adding membranes immediately after adding the radioligand to wells containing buffer plus either test compound, nonspecific, or control. The assay plates were placed on a plate shaker and shaken for thirty minutes while the reactions reached equilibrium. The plates were then filtered through Beckman GF/B filters, presoaked in 6% PEI, using a Packard Filtermate 196. Filters were washed 5× with 0.2 mL ice-cold wash buffer (5 mM Tris HCl, pH 7.4.) After filters dried, 35 μl of Microscint20 (Packard) was added to each well. The plates were then counted on a Packard TopCount to determine CPM's per well. Ki values were determined for each test compound utilizing the graphic and analytical software package, GraphPad Prism.

Test B: NK₁ FLIPR Assay using Fluo-4 Dye:

FLIPR assays are performed with a device marketed by Molecular Devices, Inc., designed to precisely measure cellular fluorescence in a high throughput whole-cell assay. (Schroeder et. al., J. Biomolecular Screening, 1(2), p 75-80, 1996).

Compounds were evaluated for potency in blocking the response of U373 cells to the NK, receptor agonist Acetyl-[Arg⁶, Sar⁹, Met(O₂)¹¹]-Substance P (ASMSP) using a FLIPR instrument.

U373 cells were loaded with Fluo-4 dye (Molecular Probes) for 45 min at 37° C. and exposed to graded concentrations of compounds for 15 min at room temperature before being challenged with 10 nM-12 nM ASMSP (an approximately EC₈₀ concentration). Responses were measured as the peak relative fluorescence after agonist addition. pIC₅₀s were calculated from eleven-point concentration-response curves for each compound.

Reagents:

Cell Culture Medium:

| | |
|---|---|
| Eagle's MEM with Earle's salts and 1- | Cellgro 10-010-CV |
| glutamine (500 mL) | |
| Non-essential amino acids, 100× (5 mL) | Cellgro 25-025-CI |
| Sodium pyruvate, 100 mM (5 mL) | Cellgro 25-000-CI |
| L-Glutamine, 200 mM (5 mL) | Cellgro 25-005-CI |
| FBS (50 mL) | Cellgro 35-010-CV |

Cell Harvesting Reagents:

| | |
|---|---|
| DPBS, 1× without Ca⁺⁺ & Mg⁺⁺ | Cellgro 21-031-CV |
| 1× Trypsin - EDTA (0.5% Trypsin, | Cellgro 25-052-CI |
| 0.53% EDTA-4Na) | |

Cell Plating medium:

| | |
|---|---|
| UltraCULTURE | BioWhittaker 12-725F |
| L-Glutamine, 200 mM (5 mL/500 mL) | Cellgro 25-005-CI |

Working Buffer:

| | |
|---|---|
| 10× Hank's balanced salt solution (100 mL/L) | Gibco 14065-056 |
| HEPES buffer 1 M (15 mL/L, [final] 15 mM) | Cellgro 25-060-CI |
| Probenecid (0.71 g dissolved in 6 mL 1 M | Sigma P-8761 |
| NaOH for 1 L, [final] 2.5 mM) | |
| DDH₂0 to 1 L, adjust pH to 7.4 with NaOH | |

Dye Solution:

Fluo-4, AM dye, Molecular Probes F-14201. 50 μg lyophilized dye is dissolved in 23 μL DMSO plus 23 μL Pluronic F-127 (Molecular Probes P-3000). The 46 μL of solubilized fluo-4 dye is then added to 10 mL of working buffer solution to provide a working dye concentration of 5 μM. Each 10 mL of diluted dye is sufficient for a 384-well-plate of cells at 25 μL per well.

Agonist:

Acetyl-[Arg⁶, Sar⁹, Met(O₂)¹¹]-Substance P (ASMSP) Stock solution of 3.33×10⁻² M. Dissolve 100 mg in 3.05 mL DMSO and store in aliquots at 4° C.

Miscellaneous:

DMSO (to dissolve compounds and for tip wash)

Cell Culture and Plating Procedures:

U373 cells were grown in cell culture medium described above (30 mL per T-150 flask) and harvested when confluent as follows. Medium was removed by aspiration and cells were washed with 12 mL DPBS, 1× without Ca++ and Mg++. The DPBS was aspirated and replaced with 3 mL trypsin-EDTA. The cells plus trypsin/EDTA were incubated about 2 minutes at room temperature, until the cells detached from the flask. The harvesting reaction was quenched by addition of 9 mL culture medium and cells were resuspended by trituration. Cells were passaged at a transfer density of 1:4 every four days. For experiments, cells were counted, pelleted by centrifugation at 400×g for 5 min and resuspended in cell plating medium at a density of 480,000 cells/mL. 25 μL of this cell suspension was added to each well of a black-walled 384-well plate (Falcon Microtest, 35 3962) using a Labsystems Multidrop 384 to give 12,000 cells per well. Plates were incubated at 37° C. overnight (minimum 15 h, maximum 23 h) before use.

Compound and Agonist Preparation:

Compounds were dissolved in DMSO at a concentration of 10 mM and 120 μL of these solutions were transferred to the first well (column 1) of each row of a 96-well, round-bottomed, polypropylene storage plate (Costar 3365). Compounds on two such plates were then serially diluted simultaneously in DMSO using a Biomek 2000. 4 μL of each dilution was transferred to a deep well plate (Beckman Coulter 267006) which had been prepared previously to contain 400 μL of freshly made working buffer in each well. Concentrations resulting from this procedure are shown in Table 1. The final compound concentrations in the assay span 11 points, between 10 μM and 0.1 nM, in half-log increments.

The contents of the deep wells were mixed, and 45 μL of each dilution were transferred—in duplicate—to a 384-well polypropylene compound loading plate (Fisher 12-565-507) so that the 384-well plate contained duplicates of each of the compounds from both 96-well plates in the concentrations shown in table 1. Columns 23 & 24 of the plate contain no compound and serve as controls. Wells A-N in columns 23 and 24 were loaded with agonist only and therefore represent the maximal response. Wells O-P in columns 23 and 24 were loaded with only buffer, no agonist, and therefore represent the minimum response.

An ASMSP agonist loading plate was made by taking stock concentration of ASMSP and diluting in working buffer to give a concentration of 3.3×10⁻⁸ M. 45 μL of this solution were transferred to all wells of a 384-well polypropylene agonist loading plate (Fisher 12-565-507) except wells 023, 024, P23 & P24 which contained buffer alone and served as unstimulated controls.

Dye Loading Cells and Adding Compound:

For each 384-well assay plate of cells, 10 mL of diluted Fluo-4 dye was prepared as stated above in the methods/reagents section. First, each 384-well cell plate was washed once with working buffer on a CCS Packard plate washer. Any remaining post-wash buffer in the wells was removed by hand and 25 μL per well of Fluo-4 dye was added using a Labsystems Multidrop 384. The cell plate was returned to a 37° C. incubator for 45 min to allow the dye to permeate the cells. After 45 min of dye loading, the cell plates were washed twice with working buffer, leaving a 30 μL volume of buffer in each well. 5 μL of compound dilutions were transferred from the compound plate to the cell plate using a PlateMate Assay plates were incubated in the presence of compound for 15 min at room temperature in the dark, and then loaded onto FLIPR.

Recording Responses in FLIPR:

After the 15 min compound pre-incubation, the plates were loaded onto the FLIPR instrument, 15 μL of ASMSP agonist was added and the cellular response to the agonist was recorded for 90 seconds. The response is measured as the peak relative fluorescence after agonist addition.

Data Analysis:

Results contained in the stat files generated by FLIPR were pasted into an Excel analysis template and, after outliers were excluded, IC₅₀ values were calculated within the template using XLfit. Individual IC₅₀ values were reported, along with pIC50. When the two IC₅₀'s obtained for a compound differed by more than 3-fold that compound was assayed one or two more times to re-determine the value.

Compound A of the present invention had a Ki of about 2 nM in Test A and an IC₅₀ of about 12 nM in Test B.

### EXAMPLES

The invention is illustrated by, but not limited to, the following examples in which descriptions, where applicable and unless otherwise stated, the following terms, abbreviations and conditions are used:
aq., aqueous; atm, atmospheric pressure; BOC, 1,1-dimethylethoxycarbonyl; DCM, dichloromethane; DMF, N,N-dimethylformamide; DMSO, dimethyl sulfoxide; EtOH, ethanol; Et2O, diethyl ether; EtOAc, ethyl acetate; h, hour(s); HPLC, high pressure liquid chromatography; HOBT, 1-hydroxybenzotriazole; MeOH, methanol; min, minutes; MS, mass spectrum; NMR, nuclear magnetic resonance; psi, pounds per square inch; RT, room temperature; sat., saturated; TEA, triethylamine; TFA, trifluoroacetic acid; THF, tetrahydrofuran.

Temperatures are given in degrees Celsius (° C.); unless otherwise stated, operations were carried out at room or ambient temperature (18-25° C.).

Organic solutions were dried over anhydrous sodium or magnesium sulfate; evaporation of solvent was carried out using a rotary evaporator under reduced pressure (4.5-30 mm Hg) with a bath temperature of up to 60° C.

Chromatography means flash column chromatography on silica gel unless otherwise noted; solvent mixture compositions are given as volume percentages or volume ratios.

When given, NMR data is in the form of delta values for major diagnostic protons (given in parts per million (ppm) relative to tetramethylsilane as an internal standard) determined at 300 MHz.

Melting points are uncorrected.

Mass spectra (MS) were obtained using an automated system with atmospheric pressure chemical ionization (APCI) unless otherwise indicated. Masses corresponding to the major isotopic component, or the lowest mass for compounds with multiple masses with nearly equivalent abundance (isotope splitting), are reported.

Where noted that a final compound was converted to the citrate salt, the free base was dissolved in methanol, DCM, or acetonitrile, combined with citric acid (1.0 equivalents) in methanol, concentrated under reduced pressure and dried in vacuo (25-60° C.). When indicated that the salt was isolated by filtration from Et₂O, the citrate salt of the compound was stirred in Et₂O for 4-18 h, recovered by filtration, washed with Et₂O, and dried in vacuo (25-60° C.).

### Example 1

### 1-N-methyl-4-(3,4-dichlorophenyl) 4-((3-cyano-2-methoxynaphth-1-yl)methoxymethyl)piperidine

The title compound of the structure below 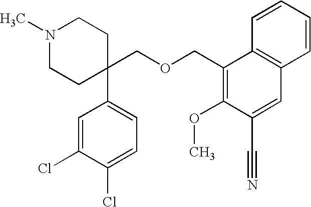
was prepared as follows. A solution containing 1-N-methyl-4-hydroxymethyl-4-(3,4-dichlorophenyl)piperidine (76.8 mg, 0.28 mmol) and dry DMF (2 mL) was cooled (ice bath) and NaH (11 mg of 60% suspension in mineral oil) was added in one portion. After 15 min, a solution containing 3-cyano-2-methoxy-1-bromomethylnaphthalene (57 mg, 0.21 mmol) and dry DMF (2 mL) was added (in 0.25 mL portions over several minutes), the mixture stirred for 15 min, allowed to warm to RT, stirred for an additional 2.5 h, then partitioned between EtOAc and water. The organic layer was separated, washed with sat. aq. NaHCO₃, dried (Na₂SO₄), filtered, and concentrated. The residue was purified by chromatography (0-5% MeOH/DCM), converted to the citrate salt, and isolated by filtration from Et₂O to give the citrate salt of the title compound as a white powder. MS m/z 469 (M+H).

The requisite 1-N-methyl-4-hydroxymethyl-4-(3,4-dichlorophenyl)piperidine was prepared as follows:

a) 1-N-methyl-4-hydroxymethyl-4-(3,4-dichlorophenyl)piperidine

To a stirred solution containing ethyl 1-N-methyl-4-(3,4-dichlorophenyl)piperidine-4-carboxylate (404 mg, 1.28 mmol) and dry Et₂O (5 mL), LiEt₃BH (1 M in THF)(4 mL) was slowly added. After 1 h at RT, a solution of 1N aq. HCl (10 mL) was slowly added, stirred for 18 h, concentrated, neutralized (sat. aq. NaHCO₃), and extracted with DCM (4×). The DCM extracts were combined, dried, filtered, and concentrated to give the title compound as a white solid. MS m/z 274 (M+H). The material was used without further purification.

b) Ethyl 1-N-methyl-4-(3,4-dichlorophenyl)piperidine-4-carboxylate

A solution containing 1-N-methyl-4-(3,4-dichlorophenyl)piperidine-4-carboxylic acid hydrochloride (550 mg, 1.69 mmol), H₂SO₄ (0.25 mL), and ethanol (25 mL) was heated under reflux for 5.5 d, cooled to RT, and concentrated. The residue was partitioned between EtOAc and sat. aq. NaHCO₃, the organic layer was separated, and the aqueous phase extracted with additional EtOAc (2×). The EtOAc extracts were combined, dried, filtered, concentrated, and the residue purified by chromatography (2% MeOH/DCM) to give the title compound as a pale-yellow oil. MS m/z 316 (M+H). ¹H NMR (CDCl₃) δ 7.47 (d, 1H), 7.39 (d, 1H), 7.22 (m, 1H), 4.14 (q, 2H), 2.77 (bd, 2H), 2.54 (bd, 2H), 2.26 (s, 3H), 2.13 (bt, 2H), 1.91 (bm, 2H), 1.2 (t, 3H).

c) 1-N-methyl-4-(3,4-dichlorophenyl)piperidine-4-carboxylic Acid Hydrochloride

A mixture containing 1-N-methyl-4-(3,4-dichlorophenyl)-4-cyanopiperidine (1.03 g, 3.83 mmol) and 8N aq. HCl (50 mL) was heated (100° C.) for 90 h, cooled to RT, and concentrated. The residue was treated with a small amount of MeOH, warmed, diluted with water and allowed to stand at RT. The solids present were isolated by filtration, washed with minimal water, and dried (60° C.) under reduced pressure to give the title compound as an off-white solid. MS m/z 288 (M+H).

d) 1-N-methyl-4-(3,4-dichlorophenyl)-4-cyanopiperidine

A mixture containing 3,4-dichlorophenylacetonitrile (4.9 g, 26.44 mmol), N-methyl-bis-(2-chloroethyl)amine hydrochloride (5.1 g, 26.49 mmol), hexadecyltributylphosphonium bromide (0.72 g, 1.43 mmol), and 50% aq. sodium hydroxide (30 mL) was heated at 100° C. for 1 hour, allowed to cool, treated with water (100 mL), and extracted with Et₂O (3×). The ether extracts were combined, washed with water (IX), and extracted with 1N aq. HCl (5×). The acidic extracts were washed (Et₂O), neutralized with solid sodium carbonate, and extracted with Et₂O (2×). The ether extracts were dried, filtered and concentrated. The residual oil was purified by chromatography (0.5-2% MeOH/DCM) to give the title compound as a yellow oil. MS m/z 269 (M+H).

The requisite 3-cyano-2-methoxy-1-bromomethylnaphthalene was prepared as follows:

a) 3-cyano-2-methoxy-1-bromomethylnaphthalene

A solution containing 3-cyano-2-methoxy-1-hydroxymethylnaphthalene (101 mg, 0.47 mmol), pyridine (0.1 mL), and dry acetonitrile (4.5 mL) was cooled (ice bath), and dibromotriphenylphosphorane (424 mg, 1.0 mmol) was added (in portions) over 5 min. After 5 min, the mixture was allowed to warm to RT, stirred for 3 h, concentrated, treated with EtOAc, and filtered. The filtrates were washed (1N aq. HCl and sat. aq. NaHCO₃), dried, filtered, and concentrated. The residue was purified by chromatography (DCM) to give the title compound as a white solid. MS m/z 276 (M+H). ¹H NMR (CDCl₃) δ 8.22 (s, 1H), 8.10 (d, 1H), 7.88 (d, 1H), 7.76 (m, 1H), 7.57 (m, 1H), 5.01 (s, 2H), 4.19 (s, 3H).

b) 3-cyano-2-methoxy-1-hydroxymethylnaphthalene

A solution containing 3-cyano-2-methoxy-1-napthoic acid (10 g, 44 mmol) and dry THF (220 mL) was cooled (ice bath), and TEA (6.5 mL, 132 mmol) and isobutylchloroformate (6.0 mL, 46.3 mmol) were added. After 30 min, the suspension was allowed to warm to RT, stirred for an additional 1.5 h, filtered into a suspension of NaBH₄ (5 g, 132 mmol) and water (200 mL), and stirred at RT for 55 h. The THF was removed, and the solids present were recovered by filtration. Following drying (50° C.) under reduced pressure, the title compound (3.12 g, 33%) was obtained as a white powder. ¹H NMR (D6-DMSO) δ 8.57 (s, 1H), 8.27 (d, J=8.4 Hz, 1H), 8.03 (d, J=8.1 Hz, 1H), 7.75 (t, J=8.1 Hz, 1H), 7.61 (t, J=7.8 Hz, 1H), 5.35 (t, J=5.4 Hz, 1H), 4.94 (d, J=5.1 Hz, 2H), 3.97 (s, 3H).

### Example 2

### 4-(4-fluorophenyl)-4-[(3-cyano-2,4-dimethoxynaphth-1-yl)methoxymethyl]piperidine

The title compound of the following structure 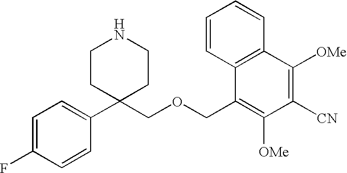
was prepared as a citrate salt as follows. To a solution containing 1-N-t-Boc-4(4-fluorophenyl)-4-hydroxymethylpiperidine (1.914 g, 6.19 mmol) in 30 mL of dry DMF was added NaH (0.272 g, 6.81 mmol) at 0° C. The solution was stirred at RT for 20 min. 3-cyano-2,4-dimethoxy-1-iodomethylnaphthalene (2.0 g, 6.19 mmol) in DMF (10 mL) was added to above solution at 0° C. The mixture was stirred at 0° C. for 20 min, RT overnight. Saturated NaHCO₃ was added and the mixture was extracted with EtOAc (3×). Combined EtOAc were washed with saturated NaCl, dried, filtered and concentrated. The residue was purified by chromatography (0.5%, 1% MeOH-DCM) to give N-t-Boc-4-(4-fluorophenyl)-4-[(3-cyano-2,4-dimethoxynaphth-1-yl)methoxymethyl]piperidine as a light yellow foaming solid (0.843 g, 27% yield). To a solution of N-t-Boc-4-(4-fluorophenyl)-4-[(3-cyano-2,4-dimethoxynaphth-1-yl)methoxymethyl]piperidine (35 mg, 0.066 mmol) in EtOAc (1 mL) at 0° C. was added HCl (37%, 0.37 mL). The solution was stirred at RT overnight and saturated NaHCO₃ was added. The mixture was extracted with DCM (2×). Combined DCM extracts were dried, filtered and concentrated. The residue was purified by chromatography (2%, 4% MeOH-DCM, 5%-8% MeOH-DCM with 1% of NH₄OH) to give the title compound as a light yellow solid (13 mg, 46% yield). MS m/z 435.5 (M+H).

The requisite 1-N-t-Boc-4(4-fluorophenyl)-4-hydroxymethylpiperidine was prepared as follows:
(a) 4-(4-fluorophenyl)-4-cyanopiperidine

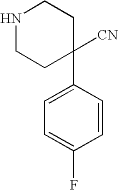

To a solution containing bis(2-chloroethyl)amine hydrochloride (6.0 g, 33.6 mmol) and 4-fluorophenyl acetonitrile (4.542 g, 33.6 mmol) in DMF (30 mL) was added sodium hydride (5.38 g, 134.4 mmol) slowly at 0° C. The resulting suspension was stirred and heated at 60° C. for 24 hrs. The reaction mixture was quenched with ice water, extracted with EtOAc (3×). The organic extracts were combined, washed with saturated NaCl (3×), dried, filtered, and concentrated. The residue was purified by chromatography (1-5% MeOH-DCM) to give the title compound as a yellow oil (2.3 g, 33% yield). MS m/z 205.38 (M+H).
(b) 4-(4-fluorophenyl)-4-carboxypiperidine hydrochloride

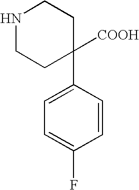

To a solution of 4-(4-fluorophenyl)-4-cyanopiperidine (4.823 g, 23.6 mmol) in ethanol (45 mL) was added water (45 mL), potassium hydroxide (19.8 g, 354 mmol). The solution was heated to 110° C. for 48 h. After cooling to RT, 37% hydrochloric acid was added to achieve pH I and solvent was removed. The residue was suspended in water (40 mL), filtered and the white solid was washed with cold water (8 mL). After drying at 50° C. for overnight, the title compound was collected as a white solid. MS m/z 224.34 (M+H).
(c) 1-N-t-Boc-4(4-fluorophenyl)-4-hydroxymethylpiperidine

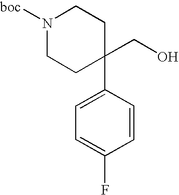

To a solution of 4-(4-fluorophenyl)-4-carboxypiperidine hydrochloride (6.134 g, 23.6 mmol) in THF (50 mL) was added 1M LAH in THF (47 mL, 47.2 mmol) at 0° C. The solution was heated to reflux for 1.5 h. The reaction was quenched by adding 2 N NaOH (2.85 mL), followed by water (3.56 mL). NaOH (0.94 g in 11.2 mL of water) was then added, followed by a solution of Boc anhydride (5.16 g, 23.6 mmol) in DCM (30 mL). The mixture was stirred at RT for overnight, filtered through diatomaceous earth, washed with EtOAc, dried, filtered and concentrated. The residue was purified by chromatography (1%, 5% MeOH-DCM) to give the title compound as a colorless oil (2.118 g, 29% yield 2 steps). MS m/z 210.40 (M+H).

### Example 3

### 4-(4-fluorophenyl)-4-[(3-cyano-2-methoxynaphth-1-yl)methoxymethyl]piperidine

A compound of the following structure 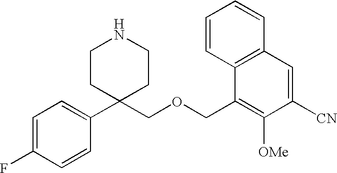
was prepared as a citrate salt via reaction procedures similar to those given in Example 1 but with replacement of 3-cyano-2,4-dimethoxy-1-iodomethylnaphthalene with 3-cyano-2-dimethoxy-1-iodomethylnaphthalene. The title compound was obtained as a light yellow solid. MS m/z 405.53 (M+H).

### Example 4

### {2-[4-(3-Cyano-2-methoxy-naphthalen-1-ylmethoxymethyl)-4-(4-fluoro-phenyl)-piperidin-1-yl]-2-oxo-ethyl}-methyl-carbamic Acid Tert-Butyl Ester

The title compound of the following structure 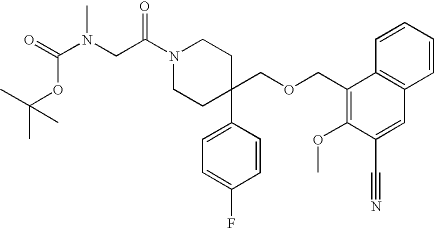
was prepared as follows. To a solution containing N-t-Boc-sarcosine (36 mg, 0.19 mmol), 4-(4-fluorophenyl)-4-[(3-cyano-2-methoxynaphth-1-yl)methoxymethyl]piperidine (70 mg, 0.17 mmol), 1-ethyl-3-(3-dimethylaminopropyl)carbodiimide hydrochloride (53 mg, 0.28 mmol) and 1-hydroxybenzotriazole (47 mg, 0.35 mmol) in DCM (5 mL) was added TEA (0.072 mL, 0.51 mmol). The solution was stirred at RT overnight. The mixture was partitioned between DCM and saturated NaHCO₃, the organic layer was removed, and the aqueous layer extracted with DCM (2×). The organic extracts were combined, dried, filtered, and concentrated. The residue was purified by chromatography (1%, 2% MeOH-DCM) to give the desired compound as a white solid (83 mg, 83% yield). MS m/z 476.48 (M+H).

### Example 5

### 4-[4-(4-Fluoro-phenyl)-1-(2-methylamino-acetyl)-piperidin-4-ylmethoxymethyl]-3-methoxy-naphthalene-2-carbonitrile

The title compound of the following structure 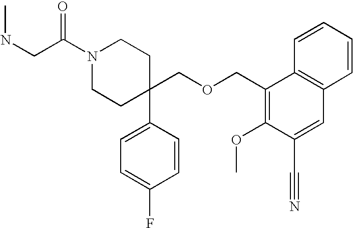
was prepared as a citrate salt as follows. To a solution of the compound of Example 4 (73 mg, 0.066 mmol) in EtOAc (2 mL) at 0° C. was added HCl (37%, 0.49 mL). The solution was stirred at RT for 1 hour and saturated NaHCO₃ was added. The mixture was extracted with DCM (3×). Combined DCM were dried, filtered and concentrated. The residue was purified by chromatography (1%, 2% MeOH-DCM, 8% MeOH-DCM with 1% of NH₄OH) to give the desired compound as a white solid (34 mg, 57% yield). MS m/z 476.51 (M+H).

### Examples 6 to 9

Compounds of the following structure

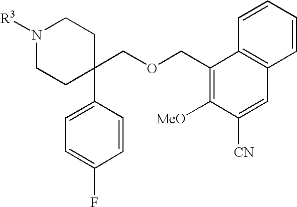

listed in Table 1, below, were prepared using procedures described in Examples 4 and 5 by replacing N-t-Boc-sarcosine with the appropriate amino acids.

TABLE 1

| Example # | R³ group | yield | MS m/z (M + H) |
|---|---|---|---|
| 4 | 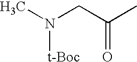 | 83% | 476.48 |
| 5 | 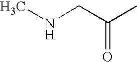 | 57% | 476.51 |
| 6 | 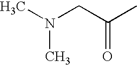 | 61% | 490.48 |
| 7 | 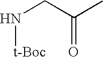 | 84% | 462.51 |
| 8 | 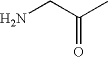 | 88% | 462.43 |
| 9 | 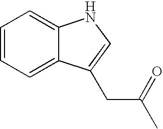 | 82% | 562.38 |

### Example 10

### 4-{4-(4-Fluoro-phenyl)-1-[2-(1H-indol-3-yl)-ethyl]-piperidin-4-ylmethoxymethyl}-3-methoxy-naphthalene-2-carbonitrile

The title compound of the following structure 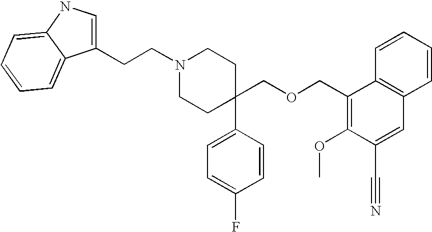
was prepared as follows. To a solution of 4-(4-fluorophenyl)-4-[(3-cyano-2-methoxynaphth-1-yl)methoxymethyl]piperidine (89 mg, 0.22 mmol) and 3-(2-bromoethyl)indole (64 mg, 0.28 mmol) in DMSO (2 mL) was added TEA (0.092 mL, 0.66 mmol). The solution was heated at 100° C. for over night. After cooling the reaction mixture was added to RT water. The mixture was extracted with EtOAc (3×). The combined EtOAc were washed with saturated NaCl, dried, filtered and concentrated. The residue was purified by chromatography (0.5%-2% MeOH-DCM) to give the desired compound as a light yellow solid (54 mg, 45% yield). MS m/z 548.55 (M+H).

### Examples 11 to 18

Compounds of the following structure

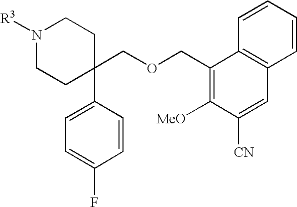

Shown in Table 2, below, were prepared using procedures described in Example 10 by replacing 3-(2-bromoethyl)indole with an appropriate halide substituted compound as shown in the table.

TABLE 2

| Example # | R³-halide¹ | Reaction Conditions | yield | MS m/z (M + H) |
|---|---|---|---|---|
| 10 | 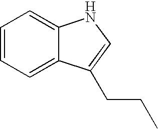 | 100° C., overnight | 45% | 548.55 |
| 11 | 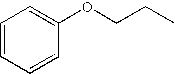 | 80° C., 3h | 63% | 525.52 |
| 12 | 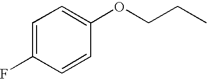 | 80° C., 3h | 60% | 543.50 |
| 13 | 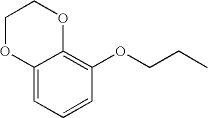 | 80° C., 3h | 44% | 583.5 |
| 14 | 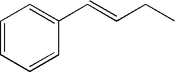 | RT, 3h | 32% | 521.46 |
| 15 | 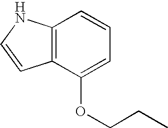 | 80° C., 6h, 100° C., 4h | 31% | 564.53 |
| 16 | 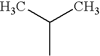 | 80° C., 6h | 25% | 447.58 |
| 17 | 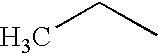 | 80° C., 7h | 27% | 433.53 |
| 18 | 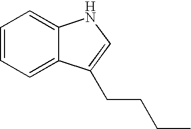 | 100° C., overnight | 45% | 562.54 |

^{1:}For Example 15 the halide is Cl and for Examples 10-14 and 16-18 the halide is Br

### Example 19

### 1-N-Methyl-4-(3,4-dichlorophenyl)-4-((3-cyanonaphth-1-yl)methoxymethyl) piperidine,

The title compound, of the structure below 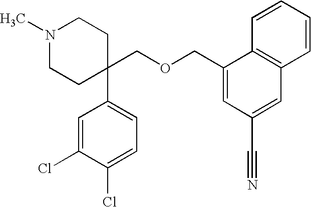
was prepared as follows. A solution containing 1-N-methyl-4-hydroxymethyl-4-(3,4-dichlorophenyl)piperidine (100 mg, 0.36 mmol) and dry DMF (3 mL) was cooled in an ice bath and NaH (14.4 mg of 60% suspension in mineral oil) was added in one portion. After 15 min, a solution containing 3-cyano-1-iodomethylnaphthalene (105 mg, 036 mmol) and dry DMF (5 mL) was added (in 0.50 mL portions over several minutes), the mixture was stirred for 15 min, allowed to warm to RT, stirred for an additional 2.5 b, then partitioned between EtOAc and water. The organic layer was separated, washed sequentially with: 1) sat. NaHCO₃, 2) sat. brine, then dried over MgSO₄, filtered, and concentrated. The residue was purified by chromatography (95:4:1 CH₂Cl₂, MeOH, NH₄OH), converted to the citrate salt, azeotroped from ether/hexane and dried at 50° C. under oil pump vacuum overnight yielding the citrate salt of the title compound as a white powder (117 mg, 53% yield). MS m/z 439 (M+H).

The requisite 1-N-methyl-4-hydroxymethyl-4-(3,4-dichlorophenyl)piperidine was prepared as described in Example 1.

The requisite 3-cyano-1-iodomethylnaphthalene was prepared as follows:

a) 3-Cyano-1-hydroxymethyl naphthalene

To a cooled solution (0° C.) containing DMF (3.75 mL, 50.8 mmol) in methylene chloride (60 mL) was added oxalyl chloride (12.7 mL, 145 mmol) dropwise. The resulting suspension was stirred at 0° C. for 1 hr. Solvent was removed in vacuo yielding a pale yellow solid. This solid was resuspended in acetonitrile (50 mL) and THF (100 mL) and cooled to 0° C. To this cooled suspension was added a solution containing 3-cyanonaphthalene-1-carboxylic acid (7.5 g, 38.1 mmol) in THF (150 mL). The reaction was stirred at 0° C. for 1.5 hours then cooled to −78° C. To this cooled solution was added, dropwise; a solution containing NaBH₄ (5.2 g, 137 mmol) in DMF (60 mL). The reaction was stirred at −78° C. for 1 hr, allowed to warm to −20° C., held at −20° C. for 2 hr, then allowed to warm to RT. Solvent was removed in vacuo. The residue was quenched with ice-cold aqueous HCl (1 N), extracted with EtOAc (2×). The organic extracts were combined, washed with: 1) HCl (1 N), 2) saturated NaCl (2×), dried, filtered, and concentrated. The residue was purified by chromatography (0-1% DCM-MeOH) to give the title compound as a yellow solid. (6.12 g, 88% yield). MS m/z fragments only.

b) 3-Cyano-1-iodomethyl naphthalene

To a solution containing 3-cyano-1-hydroxymethyl naphthalene (5.85 g, 31.97 mmol) in acetonitrile (100 mL) under nitrogen was added trimethylsilylpolyphosphate (15 mL). The reaction mixture was stirred at RT for 15 minutes. To this solution was added NaI (8.3 g, 55.2 mmol). The suspension was stirred at RT overnight. Solvent was removed in vacuo. Residue was suspended in saturated NaHCO₃ (600 mL) and extracted with ethyl acetate (2×350 mL). Combined ethyl acetate extracts were washed with: 1) saturated NaHCO₃ 2) saturated Na₂S₂O₃, 3) saturated brine, dried over MgSO₄, filtered, and concentrated. The residue was purified by crystallization from ethyl acetate yielding the title compound as a pale yellow solid (6.59 g, 70% yield). MS m/z fragments only (M+H).

### Example 20

### 1-N-Methyl-4-(3,4-dichlorophenyl)-4-((3-cyano-2,4-dimethoxynaphth-1-yl)methoxymethyl)piperidine,

The title compound, of the formula below 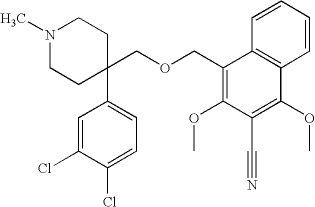
was prepared as follows. A solution containing 1-N-methyl-4-hydroxymethyl-4-(3,4-dichlorophenyl)piperidine (49 mg, 0.179 mmol) and dry DMF (2 mL) was cooled (ice bath) and NaH (7 mg of 60% suspension in mineral oil) was added in one portion. After 15 min, a solution containing 3-cyano-2,4-dimethoxy-1-iodomethylnaphthalene (63 mg, 0.178 mmol) and dry DMF (5 mL) was added (in 0.50 mL portions over several minutes), the mixture stirred for 15 min, allowed to warm to RT, stirred for an additional 2.5 h, then partitioned between EtOAc and water. The organic layer was separated, washed sequentially with: 1) sat. NaHCO₃, 2) sat. brine then, dried over MgSO₄, filtered, and concentrated. The residue was purified by chromatography (93:6:1 CH₂Cl₂, MeOH, NH₄OH), converted to the citrate salt, azeotroped from ether/hexane and dried at 50° C. under oil pump vacuum overnight yielding the citrate salt of the title compound as a white powder (57 mg, 64% yield). MS m/z 499 (M+H).

The requisite 1-N-methyl-4-hydroxymethyl-4-(3,4-dichlorophenyl)piperidine was prepared as described in Example 19.

The requisite 3-cyano-2,4-dimethoxy-1-iodomethylnaphthalene was prepared as follows:

a) 3-Cyano-2,4-dimethoxy-1-hydroxymethyl naphthalene

To a cooled solution (0° C.) containing DMF (3.75 mL, 50.8 mmol) in methylene chloride (60 mL) was added oxalyl chloride (12.7 mL, 145 mmol) dropwise. The resulting suspension was stirred at 0° C. for 1 hr. Solvent was removed in vacuo yielding a pale yellow solid. This solid was resuspended in acetonitrile (50 mL) and THF (100 mL). To this cooled suspension was added a solution containing 3-cyano-2,4-dimethoxynaphthalene-1-carboxylic acid (9.8 g, 38.1 mmol) in THF (150 mL). The reaction was stirred at 0° C. for 1.5 hours then cooled to −78° C. To this cooled solution was added, dropwise; a solution containing NaBH₄ (5.2 g, 137 mmol) in DMF (60 mL). The reaction was stirred at −78° C. for 1 hr, allowed to warm to −20° C., held at −20° C. for 2 hr, then allowed to warm to RT. Solvent was removed in vacuo. The residue was quenched with ice-cold aqueous HCl (1 N), extracted with EtOAc (2×). The organic extracts were combined, washed with: 1) HCl (1 N), 2) saturated NaCl (2×), dried, filtered, and concentrated. The residue was washed with hexane (3×) and dried in vacuo yielding the title compound as a white solid. (9.26 g, 100% yield). MS m/z fragments only.

b) 3-cyano-2,4-dimethoxy-1-iodomethylnaphthalene

To a solution containing 3-cyano-2,4-dimethoxy-1-hydroxymethylnaphthalene (2.20 g, 9.05 mmol) in acetonitrile (45 mL) under nitrogen was added trimethylsilylpolyphosphate (6 mL). Reaction was stirred at RT for 15 minutes. To this solution was added NaI (1.7 g, 11.3 mmol). Reaction was stirred for 1.5 hr. Solvent was removed in vacuo. Residue was suspended in saturated NaHCO₃ (300 mL) and extracted with ethyl acetate (2×200 mL). Combined ethyl acetate extracts were washed with: 1) saturated NaHCO₃, 2) saturated Na₂S₂O₃, 3) saturated brine dried over MgSO₄, filtered, and concentrated. The residue was purified by chromatography (3:1 DCM-hexane) to give the title compound as a yellow solid. (10.98 g, 63% yield). MS m/z fragments only.

### Example 21

### 1-N-methyl-4-(4-fluororophenyl)-4-((3-cyanonaphth-1-yl)methoxymethyl)-piperidine

The title compound, of the structure below 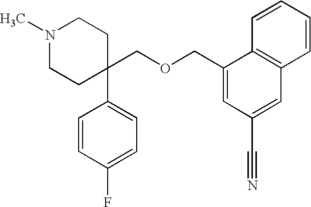
was prepared as follows. A solution containing 1-N-methyl-4-hydroxymethyl-4-(4-fluororophenyl) piperidine (100 mg, 0.45 mmol) and dry DMF (5 mL) was cooled (ice bath) and NaH (18 mg of 60% suspension in mineral oil) was added in one portion. After 15 min, a solution containing 3-cyano-1-iodomethylnaphthalene (132 mg, 0.45 mmol) and dry DMF (5 mL) was added (in 0.50 mL portions over several minutes), the mixture stirred for 15 min, allowed to warm to RT, stirred for an additional 2.5 h, then partitioned between EtOAc and water. The organic layer was separated, washed sequentially with: 1) sat. NaHCO₃, 2) sat. brine then dried over MgSO₄, filtered, and concentrated. The residue was purified by chromatography (97:2:1 CH₂Cl₂, MeOH, NH₄OH), converted to the citrate salt, azeotroped from ether/hexane and dried at 50° C. under oil pump vacuum overnight yielding the citrate salt of the title compound as a white powder (112 mg, 44% yield). MS m/z 389 (M+H).

The requisite 1-N-methyl-4-hydroxymethyl-4-(4-fluorophenyl)piperidine was prepared as follows:

a) 1-N-methyl-4-hydroxymethyl-4-(4-fluorophenyl)piperidine

To a stirred solution containing 1-N-methyl-4-(4-fluorophenyl)piperidine-4-carboxylic acid hydrochloride (1.75 g, 6.40 mmol) and dry THF (250 mL), LiAlH₄ (0.97 g, 25.6 mmol) was added in portions (0.10 g) over 10 min. The reaction mixture was heated to reflux for 2 hr. then cooled to RT. The mixture was poured slowly into HCl (1 M aqueous). Aqueous suspension was then made basic by addition of NaOH (2 M aqueous). Extract with EtOAC (2×175 mL). The combined EtOAC extracts were washed sequentially with: 1) sat. NaHCO₃, 2) sat. brine then dried (MgSO₄), filtered, and concentrated. The residue was purified by chromatography (CH₂Cl₂, 10 to 20% MeOH, 1% NH₄OH), azeotroped from ether/hexane and dried at 50° C. overnight yielding the title compound as a white powder (1.22 g, 78% yield). MS m/z 224 (M+H).

b 1-N-methyl-4-(4-fluorophenyl)piperidine-4-carboxylic acid hydrochloride

A mixture containing 1-N-methyl-4-(4-fluorophenyl)₄-cyanopiperidine (0.33 g, 1.51 mmol) and 10 N aq. HCl (5 mL) was heated in a microwave oven (power: 70 W, temp: 150° C., pressure limit: 275 psig, time: 14 min). Solvent was removed in vacuo. The residue was azeotroped from MeOH (5×) then ether (5×), dried at 50° C. under oil pump vacuum overnight yielding the title compound (0.41 g, 100% yield) as a pale tan solid. MS m/z 238 (M+H). This material was used without further purification.

c) 1-N-methyl-4-(4-fluorophenyl)-4-cyanopiperidine

The requisite 1-N-methyl-4-(4-fluorophenyl)-4-cyanopiperidine was prepared as follows:
a) 1-N-Methyl-4-(4-fluorophenyl)-4-cyanopiperidine

To a solution containing mechlorethamine hydrochloride (1.923 g, 9.99 mmol) and 4-fluorophenyl acetonitrile (1.35 g, 9.99 mmol) in DMF (30 mL) was added sodium hydride (1.6 g, 40 mmol) slowly at 0° C. The resulting suspension was stirred and heated at 60° C. for 24 hrs. The reaction mixture was quenched with ice water, extracted with EtOAc (3×). The organic extracts were combined, washed with saturated NaCl (3×), dried, filtered, and concentrated. The residue was purified by chromatography (2-5% MeOH-DCM) to give the title compound as a yellow oil (1.788 g, 82% yield). MS m/z 219.38 (M+H).

### Examples 22 to 28

The compounds shown in Table 3 were prepared by reaction procedures similar to those given in Example 1 by replacing 1-N-methyl-4-hydroxymethyl-4-(3,4-dichlorophenyl)piperidine with the appropriately substituted piperidine, and replacing 3-cyano-1-iodomethyl naphthalene with the appropriately substituted 3-cyano-1-iodomethyl naphthalene.

Compounds of the following formula and intermediates thereof of Examples 19 to 28 are listed in Table 3.

TABLE 3

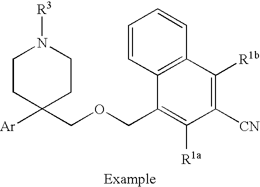

| | | |
|---|---|---|
| 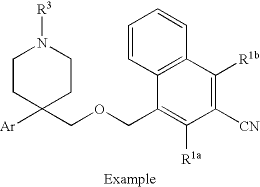 | 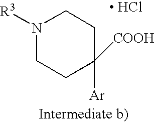 | 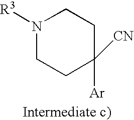 |

| Ex. # | Ar | R^{1a} | R^{1b} | R³ | Yield (%) | MS m/z (M + H) |
|---|---|---|---|---|---|---|
| 19 | 3,4-dichlorophenyl | H | H | CH₃ | 53 | 439 |
| 20 | 3,4-dichlorophenyl | OCH₃ | OCH₃ | CH₃ | 64 | 499 |
| 21 | 4-fluorophenyl | H | H | CH₃ | 44 | 389 |
| 21 a) | 4-fluorophenyl | | | CH₃ | 78 | 224 |
| 21 b) | 4-fluorophenyl | | | CH₃ | 100 | 238 |
| 21 c) | 4-fluorophenyl | | | CH₃ | 86 | 219 |
| 22 | 4-fluorophenyl | OCH₃ | OCH₃ | CH₃ | 60 | 449 |
| 23 | 4-fluorophenyl | OCH₃ | H | CH₃ | 56 | 419 |
| 24 | 4-fluorophenyl | CH₂CH₃ | H | CH₃ | 49 | 417 |
| 25 | 4-trifluoromethylfluorophenyl | H | H | CH₃ | 31 | 439 |
| 25 a) | 4-trifluoromethylfluorophenyl¹ | | | CH₃ | 70 | 274 |
| 25 b) | 4-trifluoromethylfluorophenyl² | | | CH₃ | 100 | 288 |
| 25 c) | 4-trifluoromethylfluorophenyl³ | | | CH₃ | 39 | 269 |
| 26 | 4-trifluoromethylfluorophenyl | OCH₃ | H | CH₃ | 30 | 469 |
| 27 | 4-trifluoromethylfluorophenyl | CH₂CH₃ | H | CH₃ | 28 | 467 |
| 28 | 4-trifluoromethylfluorophenyl | OCH₃ | OCH₃ | CH₃ | 20 | 499 |

^{1.}Prepared as described in example 21 a) using 1-N-methyl-4-(4-trifluoromethylphenyl)piperidine-4-carboxylic acid hydrochloride as starting material yielding

the desired 1-N-methyl-4-hydroxymethyl-4-(4-trifluoromethylphenyl)piperidine as a tan oil. MS m/z 274 (M+H). ². Prepared as described in example 21 b) using 1-N-methyl-4-(4-trifluoromethylphenyl)₄-cyanopiperidine as starting material yielding the desired 1-N-methyl-4-(4-trifluoromethylphenyl)piperidine-4-carboxylic acid hydrochloride as a tan solid. MS m/Z 288 (M+H). ³. The requisite 1-N-methyl-4-(4-trifluoromethylphenyl)-4-cyanopiperidine was prepared according to the procedure described herein.

### Example 29

### 4-{[4-Fluoro-1-napthyl)methoxy]methyl}-4-(4-fluorophenyl)-1-methylpiperidine

The title compound, of the structure below 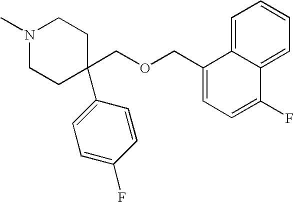
was prepared as follows.

The requisite (4-fluoro-1-napthyl)methanol was prepared as follows:

a) 4-fluoro-1-naphthoic acid (1.5 g. 7.88 mmol) and N,N-diisopropylethylamine (1.54 mL, 8.67 mmol) were dissolved in 50 mL of anhydrous THF and the solution cooled to −10° C. To the cooled solution, isobutylchloroformate (1.12 mL, 8.67 mmol) was added drop wise and stirred at for 30 minutes. The solution was filtered into a flask containing sodiumborohydride (1.19 g, 31.54 mmol) dissolved in a minimal amount of water. The foaming solution was stirred for an additional 20 minutes at RT. The solution was slowly made acidic with 1 N HCl and partitioned between EtOAc and water. The organic layer was separated and washed sequentially with 1) sat. NaHCO₃, 2) sat. brine then dried over MgSO₄ filtered, and concentrated. The residue was purified by column chromatography (1:1 EtOAc:Hexane) and the volatiles removed under reduced pressure to give the title compound as a pinkish white solid (1.0 gr, 72% yield). MS m/z 159 (M+H-H₂O)⁺

The requisite 1-(bromomethyl)₄-fluoronaphthalene was prepared as follows:

a) (4-Fluoro-1-napthyl)methanol (1.00 g, 5.67 mmol) was dissolved in 100 mL of DCM followed by the addition of carbontetrabromide (2.07 g, 6.24 mmol) and triphenylphosphine (1.63 g, 6.24 mmol) and stirred at RT overnight. The volatiles were removed under reduced pressure and the residue purified by column chromatography eluting with a gradient of 100% hexane to 100% diethyl ether. The product was obtained as a reddish brown semisolid (0.65 g, 48%).

Preparation of Title Compound:

1-(Bromomethyl)-4-fluoronaphthalene (0.33 g, 1.38 mmol) and [4-(4-fluorophenyl)-1-methylpiperdine4-yl]methanol (0.197 g, 1.24 mmol) were dissolved in 80 mL of anhydrous THF and cooled in and ice bath. To the cooled solution was added NaH (110 mg of a 60% suspension in mineral oil) was added in one portion followed by NaI (207 mg, 1.38 mmol) in one portion; the chilled solution was stirred for an additional 15 minutes. The reaction was then heated overnight to 60° C. The solution was then partitioned between EtOAc and water and the organic layer washed sequentially with 1) sat. NaHCO₃, 2) sat. brine then dried over Na₂SO₄ filtered, and concentrated. The product was purified by Mass Spec triggered HPLC fractionation (10:89:1 MeOH, Water, TFA). The TFA salt was isolated by lyophilization of the solvent and the free base prepared by passing a methanolic solution of the product through a pad of basic alumina, to provide the title compound as a translucent film (8.1 mg, 15% yield). MS m/z 382 (M+H).

### Example 30

### 4-(4-Chlorophenyl)-4-[(3-cyano-2-methoxynaphth-1-yl)methoxymethyl]piperidine

The title compound of the following structure 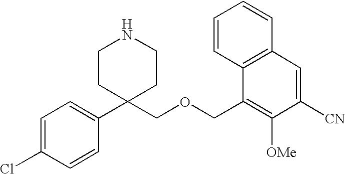
was prepared as a citrate salt, as follows. In the same manner as Example 2, but using 1-N-t-Boc4-(4-chlorophenyl)-4-hydroxymethylpiperidine (120 mg, 0.367 mmol) and 3-cyano-2-methoxy-1-bromomethylnaphthalene (102 mg, 0.369 mmol), the citrate salt was isolated by filtration from Et₂O to give the title compound (90 mg) (42%) as a white powder. MS m/z 421 (M+H).

The requisite 1-N-t-Boc-4-(4-chlorophenyl)-4-hydroxymethylpiperidine was prepared as follows:
a) 1-N-t-Boc-4-(4-chlorophenyl)₄-hydroxymethylpiperidine

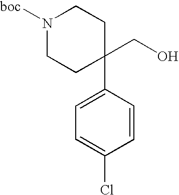

To a mixture containing 4-(4-chlorophenyl)-4-hydroxymethylpiperidine (0.46 g, 2.04 mmol), 1N aq. NaOH (5 mL), DCM (10 mL), and THF (10 mL), was added (in portions) a solution containing di-t-butyl dicarbonate (0.51 g, 2.32 mmol) and DCM (5 mL). After 72 h, the mixture was treated with sat. aq. NaHCO₃ and extracted with DCM (3×). The extracts were dried (MgSO₄), filtered, and concentrated. The residue was purified by chromatography (0-1% MeOH/DCM) to give the title compound (0.53 g) (80%) as a foamy solid. MS m/z 226 (M-BOC).
b) 4-(4-chlorophenyl)₄-hydroxymethylpiperidine

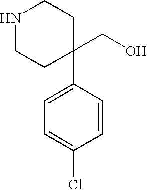

In the same manner as Example 1a, but using ethyl 4-(4-chlorophenyl)piperidine4-carboxylate (0.65 g, 2.44 mmol), the title compound (0.46 g) (83%) was obtained as an off-white solid. The material was used without further purification.
c) Ethyl 4-(4-chlorophenyl)piperidine-4-carboxylate

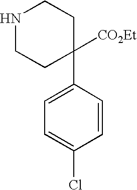

In the same manner as Example 1b, but using 4-(4-chlorophenyl)-4-carboxypiperidine hydrochloride (0.80 g, 2.91 mmol), the title compound (0.65 g) (84%) was obtained as a yellow oil. MS m/z 268 (M+H).
d) 4-(4-Chlorophenyl)-4-carboxypiperidine hydrochloride

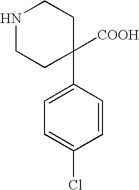

In the same manner as Example 1c, but using 4-(4-chlorophenyl)-4-cyanopiperidine (0.91 g, 4.11 mmol), the title compound (0.83 g) (73%) was obtained as an off-white solid. MS m/z 240 (M+H).
e) 4-(4-Chlorophenyl)-4-cyanopiperidine

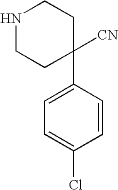

A solution containing 1-N-BOC-4-(4-chlorophenyl)-4-cyanopiperidine (1.32 g, 4.11 mmol), TFA (11 mL), and DCM (11 mL) was stirred at RT for 72 h, and then concentrated. The residue was treated with water and sat. aq. sodium bicarbonate (until basic), then extracted with DCM (3×). The DCM extracts were dried (Na₂SO₄), filtered, and concentrated to give the title compound (quant.) as a colored oil. MS m/z 221 (M+H).
f) 1-N-Boc-4-(4-chlorophenyl)-4-cyanopiperidine

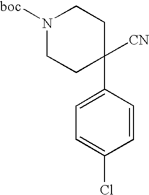

A solution containing bis(2-chloroethyl)-N-BOC amine (3.72 g, 15.38 mmol), 4-chlorobenzyl cyanide (2.10 g, 13.88 mmol), and anhydrous DMF (15 mL) was stirred and NaH (60% dispersion in mineral oil) (1.6 g, 40 mmol) was added in portions over 1 h. The mixture was heated at 60-65° C. for 1 h, stirred at RT for 72 h, then was poured into ice/water and extracted with EtOAc (2×). The organic extracts were washed (water and brine), dried, filtered, and concentrated. The residue was purified by chromatography (8:1:1 hexane/DCM/EtOAc) to give the title compound (2.4 g) (54%) as a yellow solid. MS m/z 221 (M-BOC).

### Example 31

### 1-N-methyl-4-phenyl-4-((3-cyanonaphth-1-yl)methoxymethyl)piperidine

The title compound of the following structure 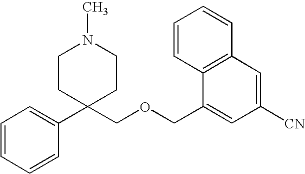
was prepared as a citrate salt, as follows. In the same manner as Example 1, but using 1-N-methyl-4-hydroxymethyl-4-phenylpiperidine (168 mg, 0.818 mmol) and 3-cyano-1-iodomethylnaphthalene (239 mg, 0.817 mmol), the citrate salt was isolated by filtration from Et₂O to give the title compound (79 mg) (17%) as an off-white powder. MS m/z 371 (M+H).

The requisite 1-N-methyl-4-hydroxymethyl-4-phenylpiperidine was prepared as follows:
a) 1-N-Methyl-4-hydroxymethyl-4-phenylpiperidine

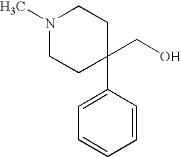

In the same manner as Example 21a, but using 1-N-methyl-4-carboxy-4-phenylpiperidine hydrochloride (1.5 g, 5.86 mmol), the title compound (1.18 g) (97%) was obtained as a white solid. MS m/z 206 (M+H).
b) 1-N-Methyl-4-carboxy-4-phenylpiperidine hydrochloride

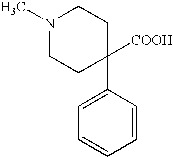

In the same manner as Example 21 b, but using 1-N-methyl-4-cyano-4-phenylpiperidine (2.0 g, 10 mmol), the title compound (1.5 g) (60%) was obtained as a white solid. MS m/z 220 (M+H).
c) 1-N-Methyl-4-cyano-4-phenylpiperidine

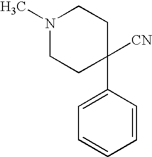

In the same manner as Example 4a, but using phenylacetonitrile (4.4 g, 37.6 mmol), and following short-path distillation, the title compound (7.05 g) (93%) was isolated as a colorless liquid. MS m/z 201 (M+H).

### Example 32

### 1-N-methyl-4-(4-fluorophenyl)₄-((naphth-1-yl)methoxymethyl) piperidine

The title compound, of the structure below 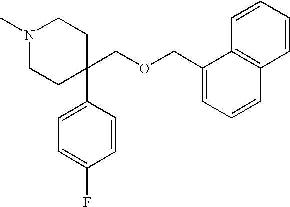
was prepared by reaction procedures similar to those given in Example 21 by replacing 3-cyano-1-iodomethylnaphthalene with 1-bromomethylnaphthalene. MS m/z364 (M+H).

### Example 33

### 1-Methyl-4-(1-naphthalen-1-yl-ethoxymethyl)-4-phenyl-piperidine

The title compound of the structure below 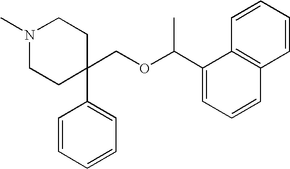
was prepared as follows. To a solution containing 1-N-methyl-4-phenyl-4-(1-naphthalen-1-yl-vinyloxymethyl)piperidine (75.0 mg, 0.21 mmol) and absolute EtOH (10 mL) was added 10% palladium on carbon (45 mg). The reaction mixture was purged with hydrogen gas (3×), then placed under hydrogen (50 psig) for 18 h. Reaction mixture was purged with nitrogen, filtered, washed with methanol (4×10 mL) and solvent removed in vacuo. The residue was then partitioned between EtOAc and sat. aq. NaHCO₃, the organic layer was separated, washed with sat. aq. NaHCO₃, dried (MgSO₄), filtered, and concentrated. The residue was converted to the citrate salt, and dried at 50° C. under oil pump vacuum yielding the title compound (72% yield, 83.5 mg) as a white powder. MS m/z 360 (M+H).

The requisite 1-N-methyl-4-phenyl-4-(1-naphthalen-1-yl-vinyloxymethyl)piperidine was prepared as follows:

a) 1-N-methyl-4-phenyl-4-(1-naphthalen-1-yl-vinyloxymethyl)piperidine

To a stirred, cooled (0° C.) solution containing naphthalene-1-carboxylic acid 1-methyl-4-phenyl-piperidin-4-ylmethyl ester (100 mg, 0.279 mmol) and dry THF (2 mL), was added Tebbe reagent (0.5M in toluene, 0.67 mL) dropwise. Reaction was stirred for 10 min, allowed to warm to RT, then stirred for an additional 1 h. Ether (10 mL) was added, and reaction was stirred for 5 min. NaOH (1M aq, 0.8 mL) was added, dropwise, and stirred for 20 min. MgSO₄(1.0 g) was added, fitered using EtOAc rinses (4×10 mL), and solvent was removed in vacuo. The residue was purified by chromatography (2% MeOH/97.6% CH₂Cl₂/0.4% NH₄OH to 7% MeOH/92.6% CH₂Cl₂/0.4% NH₄OH and concentrated to give the title compound (80% yield, 80 mg) as a white solid. MS m/z 358 (M+H).

b) Naphthalene-1-carboxylic acid 1-methyl-4-phenyl-piperidin-4-ylmethyl ester

To a suspension containing 1-naphthoic acid (445 mg, 2.58 mmol) and CH₂Cl₂ (25 mL) was added oxalyl chloride (0.293 mL, 3.35 mmol) and then DMF (2 drops). Reaction was stirred at RT for 1 h. Solvent was removed in vacuo. This material was dissolved in 1,2-dichloroethane (10 mL) and added to a solution containing 1-N-methyl-4-hydroxymethyl-4-phenylpiperidine, (0.53 g, 2.58 mmol, Example 31) triethylamine (392 mg, 3.87 mmol) and 1,2-dichloroethane (10 mL). Reaction was stirred at RT for 20 min, and then heated to 60° C. overnight. Reaction was cooled and solvent removed in vacuo. Residue was purified by chromatography (4% MeOH/95.6% CH₂Cl₂/0.4% NH₄OH) and concentrated to give the title compound (74% yield, 683 mg) as a tan solid. MS m/z 360 (M+H).

### Example 34

### 1-N-methyl-4-(4-fluorophenyl)-4-(3-cyano-1-naphthalen-1-yl-ethoxymethyl)piperidine

The title compound of the structure below 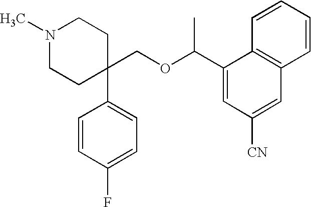
was prepared as follows. To a solution containing 1-N-methyl-4-(4-fluorophenyl)-4-(3-cyano-1-naphthalen-1-yl-vinyloxymethyl)piperidine (35.0 mg, 0.088 mmol) and absolute EtOH (10 mL) was added 10% palladium on carbon (30 mg). Reaction mixture was purged with hydrogen gas (3×), then placed under hydrogen (50 psig) for 18 h. Reaction mixture was purged with nitrogen, filtered, washed with methanol (4×10 mL) and solvent removed in vacuo. The residue was then partitioned between EtOAc and sat. aq. NaHCO₃, the organic layer was separated, washed with sat. aq. NaHCO₃, dried (MgSO₄), filtered, and concentrated. The residue was purified by chromatography (4% MeOH/95.6% CH₂Cl₂/0.4%) This chromatographed material was converted to the citrate salt, and dried at 50° C. under oil pump vacuum yielding the title compound (45% yield, 23.5 mg) as a white powder. MS m/z 403 (M+H).

The requisite 1-N-methyl-4-(4-fluorophenyl)₄-(3-cyano-1-naphthalen-1-yl-vinyloxymethyl)piperidine was prepared as follows:

a) 1-N-methyl-4-(4-fluorophenyl)-4-(3-cyano-1-naphthalen-1-yl-vinyloxymethyl)piperidine

A suspension containing 1-N-methyl-4-(4-fluorophenyl)₄-(3-bromo-1-naphthalen-1-yl-vinyloxymethyl)piperidine (123 mg, 0.27 mmol), tetrakis(triphenylphosphine) palladium(0) (9.2 mg, 0.0078 mmol), copper (1) iodide (32.7 mg, 0.172 mmol) and DMF (3 mL) was purged vigorously with nitrogen for 5 min. Reaction was microwaved for 7 min 30 sec. to 200° C. with a power: of 230 watts. Reaction was cooled, poured into sat. aq. NaHCO₃, and extracted with EtOAC (2×75 mL). The combined EtOAC extracts were washed with: 1.) sat. aq. NaHCO₃ (35 mL), 2.) sat. brine (35 mL), dried over MgSO₄, filtered and solvent removed in vacuo. The residue was purified by chromatography (4% MeOI/95.6% CH₂Cl₂/0.4% NH₄OH) yielding the title compound (39% yield, 42 mg) as a white film. MS m/z 401 (M+H).

b) 1-N-methyl-4-(4-fluorophenyl)-4-(3-bromo-1-naphthalen-1-yl-vinyloxymethyl)piperidine

To a stirred, cooled (0° C.) solution containing 3-bromo-naphthalene-1-carboxylic acid 1-methyl-4-(4-fluoro-phenyl)-piperidin-4-ylmethyl ester(310 mg, 0.68 mmol) and dry THF (12 mL), was added Tebbe reagent (0.5M in toluene, 1.71 mL) dropwise. Reaction was stirred for 10 min, allowed to warrn to RT, then stirred for an additional 1 h. Ether (20 mL) was added, and reaction was stirred for 5 min. NaOH (1M aq, 1.6 mL) was added, dropwise, and stirred for 20 min. MgSO₄(2.0 g) was added, filtered using EtOAc rinses (4×20 mL), and solvent was removed in vacuo. The residue was purified by chromatography (3% MeOH/96.6% CH₂Cl₂/0.4% NH₄OH) and concentrated to give the title compound (65% yield, 200 mg) as a yellow solid. MS m/z 454 (M+H).

c) 3-Bromo-naphthalene-1-carboxylic acid 1-methyl-4-(4-fluoro-phenyl)-piperidin-4-ylmethyl ester.

To a suspension containing 3-bromo-1-naphthoic acid (300 mg, 1.19 mmol) and CH₂Cl₂ (15 mL) was added oxalyl chloride (0.15 mL, 1.72 mmol) and then DMF (2 drops). Reaction was stirred at RT for 1 h. Solvent was removed in vacuo. This material was dissolved in 1,2-dichloroethane (10 mL) and added to a solution containing 1-N-methyl-4hydroxymethyl-4-(4-fluoro-phenyl)piperidine (261 mg, 1.17 mmol), triethylamine (182 mg, 1.8 mmol) and 1,2-dichloroethane (18 mL). Reaction was stirred at RT for 20 min, and then heated to 60° C. overnight. Reaction was cooled and solvent removed in vacuo. Residue was purified by chromatography (5% MeOH in CH₂Cl₂) and concentrated to give the title compound (63% yield, 341 mg) as a white solid. MS m/z 456 (M+H).

The required 3-bromo-1-naphthoic acid was prepared as follows using the procedure of Rule, H G and Thompson, S B; J. Chem. Soc. 1764-1767 (1937). 1,8-Naphthalic anhydride was brominated and converted to 3-bromo-1-naphthoic acid.

### Example 35

### 1-N-methyl-4-(4-fluorophenyl)-4-(3-cyano-2,4-dimethoxy-1-naphthalen-1-yl-ethoxymethyl)piperidine

The title compound of the structure below 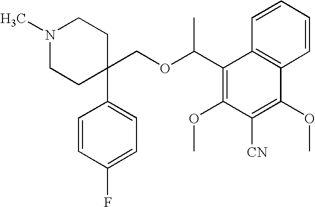
was prepared as follows. To a solution containing 1-N-methyl-4-(4-fluorophenyl)-4-(3-cyano-2,4-dimethoxy-1-naphthalen-1-yl-vinyloxymethyl)piperidine (69 mg, 0.15 mmol) and absolute EtOH (10 mL) was added 10% palladium on carbon (70 mg). Reaction mixture was purged with hydrogen gas (3×), then placed under hydrogen (50 psig) for 38 h. Reaction mixture was purged with nitrogen, filtered, washed with methanol (4×20 mL) and solvent removed in vacuo. The residue was then partitioned between EtOAc and sat. aq. NaHCO₃, the organic layer was separated, washed with sat. aq. NaHCO₃, dried (MgSO₄), filtered, and concentrated. The residue was purified by chromatography (3% MeOH/96.6% CH₂Cl₂/0.4% NH₄OH) This chromatographed material was converted to the citrate salt, and dried at 50° C. under oil pump vacuum yielding the title compound (40% yield, 39 mg) as a tan powder. MS m/z 463 (M+H).

The requisite 1-N-methyl-4-(4-fluorophenyl)-4-(3-cyano-2,4-dimethoxy-1-naphthalen-1-yl-vinyloxymethyl)piperidine was prepared as follows:

a) 1-N-methyl-4-(4-fluorophenyl)-4-(3-cyano-2,4-dimethoxy-1-naphthalen-1-yl-vinyloxymethyl)piperidine

A suspension containing 1-N-methyl-4-(4-fluorophenyl)-4-(3-iodo-2,4-dimethoxy-1-naphthalen-1-yl-vinyloxymethyl)piperidine (140 mg, 0.25 mmol), copper (1) cyanide(44.8 mg, 0.50 mmol) and DMF (6 mL) was purged vigorously with nitrogen for 5 min. Reaction was microwaved for 5 min at 150° C. at a power of 150 watts. Reaction was cooled, poured into sat. aq. NaHCO₃, and extracted with EtOAC (2×75 mL). The combined EtOAC extracts were washed with: 1.) sat. aq. NaHCO₃ (35 mL), 2.) sat. brine (35 mL), dried over MgSO₄, filtered and solvent removed in vacuo. The residue was purified by chromatography (2% MeOH/97.6% CH₂Cl₂/0.4% NH₄OH) yielding the title compound (66% yield, 76 mg) as a white film. MS m/z 461 (M+H).

b) 1-N-methyl-4-(4-fluorophenyl)₄-(3-iodo-2,4-dimethoxy-1-naphthalen-1-yl-vinyloxymethyl)piperidine.

To a stirred, cooled (0° C.) solution containing 3-iodo-2,4-dimethoxy-naphthalene-1-carboxylic acid 1-methyl-4-(4-fluoro-phenyl)-piperidin-4-ylmethyl ester(290 mg, 0.515 mmol) and dry THF (15 mL), was added Tebbe reagent (0.5 M in toluene, 2.0 mL) dropwise. Reaction was stirred for 10 min, allowed to warm to RT, then stirred for an additional 5 h. Ether (40 mL) was added, and reaction was stirred for 5 min. NaOH (1M aq, 2.0 mL) was added, dropwise, and stirred for 20 min. MgSO₄ (4.0 g) was added, fitered using EtOAc rinses (4×30 mL), and solvent was removed in vacuo. The residue was purified by chromatography (2% MeOH/97.6% CH₂Cl₂/0.4% NH₄OH to 4% MeOH/95.6% CH₂Cl₂/0.4% NH₄OH) and concentrated to give the title compound (66% yield, 201 mg) as a tan solid. MS m/z 562 (M+H).

c) 3-Iodo-2,4-dimethoxy-naphthalene-1-carboxylic acid 1-methyl-4-(4-fluoro-phenyl)-piperidin-4-ylmethyl ester.

To a solution containing 3-iodo-2,4-dimethoxy-1-naphthoic acid (855 mg, 2.39 mmol) and CH₂Cl₂ (25 mL) was added oxalyl chloride (0.30 mL, 3.44 mmol) and then DMF (3 drops). Reaction was stirred at RT for 1 h. Solvent was removed in vacuo. This material was dissolved in 1,2-dichloroethane (20 mL) and added to a solution containing 1-N-methyl-4-hydroxymethyl-4-(4-fluoro-phenyl)piperidine (522 mg, 2.34 mmol), triethylamine (364 mg, 3.6 mmol) and 1,2-dichloroethane (20 mL). Reaction was stirred at RT for 20 min, and then heated to 60° C. overnight. Reaction was cooled and solvent removed in vacuo. Residue was purified by chromatography (4% MeOH/95.6% CH₂Cl₂/0.4% NH₄OH) and concentrated to give the title compound (40% yield, 524 mg) as a white solid. MS m/z 564 (M+H).

d) 3-Iodo-2,4 dimethoxy-naphthalene-1-carboxylic Acid.

To a solution containing 3-iodo-2,4-dimethoxynaphthalene-1-carboxylic acid benzyl ester (1.20 g, 2.68 mmol) and acetonitrile (20 mL) was added trimethylsilyl iodide (0.495 mL, 3.48 mmol) dropwise. Reaction was stirred at RT for 48 h. Reaction was poured into a solution containing sat. aq. NaHCO₃ (70 mL) and sat. Na₂S₂O₃ (30 mL). The pH was adjusted to 2.0 by careful addition of 6M HCl. The acidic aqueous suspension was extracted with EtOAC (2×75 mL). The combined EtOAC extracts were washed with: 1.) 1M HCl. (35 mL), 2.) sat. brine (35 mL), dried over MgSO₄, filtered and solvent removed in vacuo. The residue was suspended in a solution containing ether (35 mL) and hexane (120 mL). Remove solvent in vacuo, dry at 50° C. under oil pump vacuum yielding the title compound (83% yield, 800 mg) as a tan powder. ¹H NMR (CDCL₃) δ 11.91 (s, 1H), 8.10 (m, 2H), 7.58 (dd, 1H), 7.49 (dd, 1H), 4.06 (s, 3H), 4.01 (s, 3H).

The required 3-iodo-2,4-dimethoxynaphthalene-1-carboxylic acid benzyl ester was prepared as follows:

a) Benzyl 2,4-dihydroxy-1-naphthoate

A mixture of dibenzyl malonate (51 mL, 204 mmol), 2-bromoacetophenone (20.25 g, 102 mmol), cupric bromide (1.60 g, 11.2 mmol) and dioxane (75 mL) was stirred at room temperature under nitrogen for 15 minutes and then treated cautiously with portionwise 60% sodium hydride (9.76 g, 244 mmol). After addition of the sodium hydride the mixture was stirred in an 80° C. oil bath for 3 h., cooled and poured onto water (1 L). Ethyl acetate (500 mL) and 1N HCl (50 mL) was added, the mixture shaken and the layers separated. The aqueous phase was extracted with a second portion of ethyl acetate (200 mL). The dried (MgSO₄) organic phases were filtered and the solvent removed in vacuo. Column chromatography (10%, 20%, 30% and 40% diethyl ether/hexane) returned a product that upon treatment with petroleum ether and dichloromethane gave 11.72 g of the yellow title compound. The material from the liquors was re-chromatographed (dichloromethane) and the collected fractions treated with 1: I petroleum ether/dichloromethane to yield additional title compound (4.80 g). Total yield was 16.52 g (55%), mp 159-162° C. ¹H NMR (300 MHz, CDCl₃) δ 5.54 (s, 2H), 5.98 (s, 1H, exchangeable), 6.50 (s, 1H), 7.32-7.54 (m, 7H), 8.17 (d, 1H, J=8.5 Hz), 8.82 (d, 1H, J=8.9 Hz), 12.72 (s, 1H, exchangeable). MS APCI, m/z=295 (M+1).

b) Benzyl 2,4-dihydroxy-3-iodo-1-naphthoate

A solution of benzyl 2,4-dihydroxy-1-naphthoate (58.86 g, 200 mmol) in acetonitrile (600 mL), warmed slightly to dissolve the material, was treated rapidly with a solution of N-iodosuccinimide (46.12 g, 205 mmol) in acetonitrile (400 mL), the mixture stirred at room temperature for 2 h and the solvent stripped in vacuo. The residue was dissolved in dichloromethane (1 L), washed with water (300 mL) containing a small amount of NaHSO₃ and the organic layer collected, dried (MgSO₄) and the solvent removed in vacuo. Column chromatography (1:1 hexane/dichloromethane) returned, after drying in vacuo, the title compound (68.13 g, 81%) as a cream colored solid. ¹H NMR (300 MHz, CDCl₃) δ 5.56 (s, 2H), 6.45 (s, 1H), 7.35-7.57 (m, 7H), 8.27 (d, 1H, J=8.3 Hz), 8.80 (d, 1H, J=8.8 Hz), 13.67 (s, 1H).

c) 3-Iodo-2,4-dimethoxynaphthalene-1-carboxylic Acid Benzyl Ester

A stirred mixture of benzyl 2,4-dihydroxy-3-iodo-1-naphthoate (68.13 g, 162 mmol), dimethyl sulfate (32 mL, 338 mmol), potassium carbonate (48 g, 347 mmol) and acetone (875 mL), under nitrogen, was refluxed overnight. The acetone was removed in vacuo and the residue was partitioned between water (500 mL) and ethyl acetate (500 mL). The separated aqueous layer was extracted with additional ethyl acetate (200 mL) and the combined organics dried (MgSO₄), filtered and the solvent removed in vacuo. Column chromatography (1:1 hexane/dichloromethane) returned the title material as a yellow oil that solidified on standing (60.48 g, 83%), mp 80-83° C. ¹H NMR (300 MHz, CDCl₃) δ 3.85 (s, 3H), 3.98 (s, 3H), 5.51 (s, 2H), 7.32-7.54 (m, 7H), 7.76 (d, 1H, J=9.7 Hz), 8.09 (d, 1H, J=9.2 Hz).

### Example 36

Following conventional procedures well known in the pharmaceutical art, the following representative pharmaceutical dosage forms may be prepared containing a compound such as Compound A in accord with formula I:

| Tablet | mg/tablet |
|---|---|
| Compound in accord with formula I | 50.0 |
| Mannitol, USP | 223.75 |
| Croscarmellose sodium | 60 |
| Maize starch | 15 |
| Hydroxypropylmethylcellulose (HPMC), USP | 2.25 |
| Magnesium stearate | 3.0 |
| Capsule | mg/capsule |
| Compound in accord with formula I | 10.0 |
| Mannitol, USP | 488.5 |
| Croscarmellose sodium | 15 |
| Magnesium stearate | 1.5 |

The pharmaceutical dosage form is administered to a patient in need thereof at a frequency depending on the patient and the precise disease condition being treated.

1. A compound in accord with formula I: 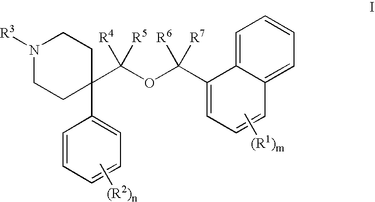
wherein:
R¹ at each occurrence is a moiety independently selected from CN, CF₃, OCF₃, OCHF₂, halogen, C₁₋₄alkyl, C₂₋₄alkenyl, C₂₋₄alkynyl, R^{a}, R^{b}, SR^{a}, NR^{e}R^{f}, CH₂NR^{e}R^{f}, OR^{c}, and CH₂OR^{c}, where m is selected from 0, 1, 2 or 3; wherein R^{a}, R^{b}, and R^{c} are independently at each occurrence selected from hydrogen, C₁₋₆alkyl, C(O)R^{d}, C(O)NHR^{d} and CO₂R^{d}, or R^{a} and R^{b} may together be (CH₂)ⱼG(CH₂)ₖ or G(CH₂)ⱼG where G is oxygen, j is 1, 2, 3 or 4, k is 0, 1 or 2; where Rd at each occurrence is independently selected from C₁₋₆alkyl, and Re and R are independently at each occurrence selected from hydrogen, C₁₋₆alkyl, C(O)R^{d}, C(O)NHR^{d}, CO₂R^{d};
R² at each occurrence is independently selected from hydrogen, CN, CF₃, OCF₃, OCHF₂, halogen, C₁₋₄alkyl, C₂₋₄alkenyl, C₂₋₄alkynyl, R^{a}, R^{b}, SR^{a}, NR^{e}R^{f}, CH₂NR^{e}R^{f}, OR^{c}, and CH₂OR^{c}, where n is selected from 0, 1, 2 or 3; wherein R^{a}, R^{b}, and R^{c} are independently at each occurrence selected from hydrogen, C₁₋₆alkyl, C(O)R^{d}, C(O)NHR^{d} and CO₂R^{d}, or R^{a} and R^{b} may together be (CH₂)ⱼG(CH₂)ₖ or G(CH₂)ⱼG where G is oxygen, j is 1, 2, 3 or 4, k is 0, 1 or 2; where R^{d} at each occurrence is independently selected from C₁₋₆alkyl, and R^{e} and R^{f} are independently at each occurrence selected from hydrogen, C₁₋₆alkyl, C(O)R^{d}, C(O)NHR^{d}, CO₂R^{d};
R³ is selected from hydrogen, C₁₋₆alkyl, C(O)—(CH₂)_{q}—NR⁸R⁹, (CH₂)ᵣ—NR⁸R⁹, (CH₂)_{q}—O-D, (CH₂)_{q}-D and (CH₂)_{q}—CH═CH-D, wherein R⁸ and R⁹ are independently selected from hydrogen and C₁₋₆alkyl, q is selected from 1, 2 or 3, r is selected from 1, 2, 3 or 4 and D is selected from phenyl or indolyl which phenyl or indolyl may bear one or more substituents selected from halogen, C₁₋₆alkyl, C₁₋₆alkoxy and —O—(CH₂)_{q}—O—;
R⁴, R⁵, R⁶ and R⁷ at each occurrence are independently selected from hydrogen or C₁₋₆alkyl, or
independently, R⁴ and R⁵ together with the carbon to which they are attached and R⁶ and R⁷ together with the carbon to which they are attached form a moiety in accord with formula II, 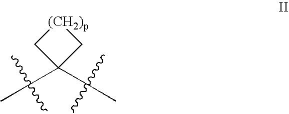
wherein p is selected from 0, 1, 2, 3 or 4;
in vivo-hydrolysable precursors thereof, and pharmaceutically-acceptable salts thereof.

2. A compound according to claim 1, wherein:
R¹ at each occurrence is independently selected from fluoro, cyano, C₁₋₆alkyl and C₁₋₆alkoxy and m is 1, 2 or 3;
R² at each occurrence is independently selected from halogen where n is 1 or 2, and
R³ is selected from hydrogen and C₁₋₆alkyl;
in vivo-hydrolysable precursors thereof, and pharmaceutically-acceptable salts thereof.

3. A compound according to claim 1, wherein:
R¹ at each occurrence is independently selected from fluoro, cyano, ethyl and methoxy and m is 1, 2 or 3;
R² at each occurrence is independently selected from halogen where n is 1 or 2, and
R³ is selected from hydrogen and methyl;
in vivo-hydrolysable precursors thereof, and pharmaceutically-acceptable salts thereof.

4. A compound according to claim 1, wherein R⁴, R⁵ and R⁶ are each hydrogen and R⁷ is methyl in vivo-hydrolysable precursors thereof, and pharmaceutically-acceptable salts thereof.

5. A compound according to claim 1, wherein:
R¹ at each occurrence is independently selected from fluoro, cyano, C₁₋₆alkyl and C₁₋₆alkoxy and m is 1, 2 or 3;
R² at each occurrence is independently selected from halogen where n is 1 or 2, and
R³ is selected from hydrogen, C₁₋₆alkyl, C(O)—(CH₂)_{q}—NR⁸R⁹, (CH₂)ᵣ—NR⁸R⁹, (CH₂)_{q}—O-D, wherein R⁸ and R⁹ are independently selected from hydrogen, C₁₋₆alkyl and C₁₋₆alkoxy, q is 1, 2 or 3, r is 1, 2, 3 or 4 and D is selected from phenyl, indol-3-yl, indol-4-yl which phenyl may bear one or more substituents selected from fluoro, methyl, ethyl, methoxy, ethoxy or —O—(CH₂)₂—O— and which indolyl may bear one or more substituents selected from fluoro, methyl, ethyl, methoxy or ethoxy, in vivo-hydrolysable precursors thereof, and pharmaceutically-acceptable salts thereof.

6. A pharmaceutical composition comprising a compound according to claim 1 together with at least one pharmaceutically-acceptable excipient or diluent.

7. The pharmaceutical composition of claim 6 for treating a disorder or condition selected from hypertension, depression, depression in cancer patients, depression in Parkinson's patients, postmyocardial infarction depression, subsyndromal symptomatic depression, depression in infertile women, pediatric depression, major depression, single episode depression, recurrent depression, child abuse induced depression, post partum depression, generalized anxiety disorder, phobias, agoraphobia, social phobia, simple phobias, posttraumatic stress syndrome, avoidant personality disorder, premature ejaculation, eating disorders, anorexia nervosa, bulimia nervosa, obesity, chemical dependencies, addictions to alcohol, cocaine, heroin, phenobarbital, nicotine or benzodiazepines, cluster headache, migraine, pain, Alzheimer's disease, obsessive-compulsive disorder, panic disorder, memory disorders, dementia, amnestic disorders, age-related cognitive decline, Parkinson's diseases, dementia in Parkinson's disease, neuroleptic-induced parkinsonism, tardive dyskinesias, endocrine disorders, hyperprolactinaemia, vasospasm, spasm of the cerebral vasculature, cerebellar ataxia, gastrointestinal tract disorders involving changes in motility and secretion, negative symptoms of schizophrenia, premenstrual syndrome, fibromyalgia syndrome, stress incontinence, Tourette's syndrome, trichotillomania, kleptomania, male impotence, attention deficit hyperactivity disorder, chronic paroxysmal hemicrania and headache associated with vascular disorders, in a mammal, preferably a human, comprising an effective amount of a compound according to claim 1 or a pharmaceutically-acceptable salt thereof effective in treating such disorder or condition and a pharmaceutically-acceptable carrier.

8. A method of treating a disease condition wherein antagonism of NK₁ receptors in combination with SRI activity is beneficial which method comprises administering to a warm-blooded animal an effective amount of a compound according to claim 1 or an in vivo-hydrolysable precursor or a pharmaceutically-acceptable salt thereof.

9. A method of treating an individual suffering from a disease condition wherein antagonism of NK₁ receptors in combination with SRI activity is beneficial which method comprises administering to a warm-blooded animal an effective amount of a compound according to claim 1 or an in vivo-hydrolysable precursor or a pharmaceutically-acceptable salt thereof.

10-11. (canceled)

12. The method according to claim 8 for treating conditions selected from hypertension, depression, depression in cancer patients, depression in Parkinson's patients, postmyocardial infarction depression, subsyndromal symptomatic depression, depression in infertile women, pediatric depression, major depression, single episode depression, recurrent depression, child abuse induced depression, post partum depression, generalized anxiety disorder, phobias, agoraphobia, social phobia, simple phobias, posttraumatic stress syndrome, avoidant personality disorder, premature ejaculation, eating disorders, anorexia nervosa, bulimia nervosa, obesity, chemical dependencies, addictions to alcohol, cocaine, heroin, phenobarbital, nicotine or benzodiazepines, cluster headache, migraine, pain, Alzheimer's disease, obsessive-compulsive disorder, panic disorder, memory disorders, dementia, amnestic disorders, age-related cognitive decline, Parkinson's diseases, dementia in Parkinson's disease, neuroleptic-induced parkinsonism, tardive dyskinesias, endocrine disorders, hyperprolactinaemia, vasospasm, spasm of the cerebral vasculature, cerebellar ataxia, gastrointestinal tract disorders involving changes in motility and secretion, negative symptoms of schizophrenia, premenstrual syndrome, fibromyalgia syndrome, stress incontinence, Tourette's syndrome, trichotillomania, kleptomania, male impotence, attention deficit hyperactivity disorder, chronic paroxysmal hemicrania and headache associated with vascular disorders, in a mammal, preferably a human, comprising administering an effective amount of a compound according to claim 1 or a pharmaceutically-acceptable salt thereof effective in treating such disorder or condition.

13. The method according to claim 9 for treating conditions selected from hypertension, depression, depression in cancer patients, depression in Parkinson's patients, postmyocardial infarction depression, subsyndromal symptomatic depression, depression in infertile women, pediatric depression, major depression, single episode depression, recurrent depression, child abuse induced depression, post partum depression, generalized anxiety disorder, phobias, agoraphobia, social phobia, simple phobias, posttraumatic stress syndrome, avoidant personality disorder, premature ejaculation, eating disorders, anorexia nervosa, bulimia nervosa, obesity, chemical dependencies, addictions to alcohol, cocaine, heroin, phenobarbital, nicotine or benzodiazepines, cluster headache, migraine, pain, Alzheimer's disease, obsessive-compulsive disorder, panic disorder, memory disorders, dementia, amnestic disorders, age-related cognitive decline, Parkinson's diseases, dementia in Parkinson's disease, neuroleptic-induced parkinsonism, tardive dyskinesias, endocrine disorders, hyperprolactinaemia, vasospasm, spasm of the cerebral vasculature, cerebellar ataxia, gastrointestinal tract disorders involving changes in motility and secretion, negative symptoms of schizophrenia, premenstrual syndrome, fibromyalgia syndrome, stress incontinence, Tourette's syndrome, trichotillomania, kleptomania, male impotence, attention deficit hyperactivity disorder, chronic paroxysmal hemicrania and headache associated with vascular disorders, in a mammal, preferably a human, comprising administering an effective amount of a compound according to claim 1 or a pharmaceutically-acceptable salt thereof effective in treating such disorder or condition.
